(19) United States
(12) Patent Application Publication
(10) Pub. No.: US 2018/0000396 A1
(43) Pub. Date: Jan. 4, 2018

(54) METHODS AND SYSTEMS FOR HYBRID OVERSIGHT OF SAMPLE COLLECTION

(71) Applicant: Theranos, Inc., Palo Alto, CA (US)

(72) Inventor: Sunny Balwani, Atherton, CA (US)

(21) Appl. No.: 15/592,935

(22) Filed: May 11, 2017

Related U.S. Application Data
(63) Continuation of application No. PCT/US2015/062741, filed on Nov. 25, 2015.
(60) Provisional application No. 62/085,229, filed on Nov. 26, 2014.

(51) Int. Cl. A61B 5/15; G09B 5/04; A61B 10/00; A61B 10/02; A61B 5/11; A61B 5/00; G09B 19/00; G09B 5/02

(52) U.S. Cl. CPC A61B 5/150748; G09B 19/00; G09B 5/04; G09B 5/02; A61B 10/02; A61B 5/1127; A61B 5/746; A61B 5/6806; A61B 5/150755; A61B 5/150076; A61B 10/007

(57) ABSTRACT

Methods for obtaining a sample from a subject include providing a sample collection room. Samples may be small, e.g., a finger-stick. A method is provided for sample collection comprising: obtaining, in a sample collection room, a sample from said body part of said subject; monitoring activity in the sample collection room using at least two motion capture devices positioned to view a field of interest in the sample collection room; and alerting a technician performing the sample collection if a non-conforming action by the technician is detected by at least one of the motion capture devices.

### BACKGROUND

Clinical samples are useful in many situations, including being useful for monitoring the health of subjects, for diagnosing diseases or pathological conditions, and for monitoring the progress of therapeutic interventions, among various uses.

Obtaining clinical samples is desirable for such uses; however, subjects may find providing clinical samples, such as blood, urine, or tissue swab samples, to be painful, or difficult, or inconvenient. Sometimes, the difficulty in sample collection and in the quality of the sample collected is attributed at least in part to the technique(s) used by the technician for the sample collection process.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Improved methods for obtaining clinical samples include providing a designated location in a point-of-service (POS) site where a clinical sample may be provided for analysis or processing. In embodiments, the sample collection process at a POS site can be monitored for quality purposes. In embodiments, improved methods for obtaining clinical samples include providing a designated location in a retail store, where the designated location is configured for obtaining a clinical sample; and obtaining a clinical sample from a subject. In embodiments, the sample may be analyzed at the same location; or at an adjacent location; or may be sent to a different location for analysis. In embodiments, the sample may be analyzed by an automatic analysis device or system at the same location; or at an adjacent location; or at a different location. A designated location in a retail store may be a sample collection room.

In embodiments, the designated location in the retail store comprises a room that is separate from other areas in the retail store; for example, the designated location may be separated from other areas in the retail store by a wall having a door, while being accessible to subjects from within the retail store. In embodiments, the designated location in the retail store comprises a room that is adjacent to, while separate from other areas in the retail store, that is accessible to subjects from outside the retail store. In embodiments, the designated location in the retail store comprises a plurality of rooms that are separate from other areas in the retail store; for example, at least one of such rooms may be accessible directly from other areas in the retail store (e.g., via a door or entry-way), or at least one of such rooms may be adjacent the other areas of the retail store and be accessible from outside the retail store. For example, such rooms may include a waiting room, a sample collection room, a bathroom, a sample analysis room, and other rooms. In embodiments, a room may serve multiple functions (e.g., a bathroom may serve as a sample collection room where a urine sample is collected; or a sample collection room may serve as a sample analysis room where a sample analysis device or a sample analysis system is located in the room where a sample is collected).

In one embodiment as described herein, a method is provided for obtaining a sample from a subject. The method comprises obtaining, in a sample collection room, a sample from a body part of the subject; and monitoring activity in the sample collection room using at least one motion capture device positioned to view the sample collection room.

In another embodiment as described herein, a method is provided for obtaining a sample from a subject. The method comprises providing a sample collection room in a retail store; obtaining, in the sample collection room, a sample from the body part of the subject; monitoring activity in the sample collection room using at least one motion capture device; and providing real-time feedback to a technician performing the sample collection technique based on motion recorded by the at least one motion capture device.

It should be understood that one or more of the following features may be adapted for use with at least one or more of the embodiments described herein. For example, the method may include warming the body part of the subject prior to and/or during the sample obtaining step. Optionally, the warming of the body part comprises warming with a warming table; a warming plate; a finger warmer; a fingertip warmer; an air-warmer; furniture for seating comprising a warming plate or other heating element; or a combination thereof. Optionally, the method comprises providing a bathroom adjacent the sample collection room; and providing a pass-through connecting the sample collection room with the bathroom, wherein the pass-through is configured to allow the transfer of a sample from the bathroom to the sample collection room. Optionally, the sample comprises a urine sample. Optionally, the method comprises using both time and position to determine if a device is being properly utilized for sample collection. Optionally, the method comprises, all, one or more of the following: providing a warming table in the sample collection room; providing a warming plate in the sample collection room; providing a fingertip warmer in the sample collection room; providing an air-warmer in the sample collection room; providing a chair or other furniture for seating in the sample collection room, the chair or other furniture for seating comprising a warming plate or other heating element; providing a reclining chair in the sample collection room, the reclining chair being configured for positioning a subject effective for obtaining a sample; providing a reclining chair in the sample collection room, the reclining chair comprising a warming plate or other heating element, the reclining chair being configured for warming at least a portion of a body part of a subject, and for positioning a subject effective for obtaining a sample; and providing a calming feature in the sample collection room.

Optionally, the calming feature is selected from the group of calming features consisting of flowing water, a flower, a plant, a calming scent, a calming sound, a calming image, and a calming color. Optionally, the calming feature is selected from the group of calming features comprising flowing water, a flower, a plant, a calming scent, a calming sound, a calming image, and a calming color. Optionally, the method comprises providing a reclining chair in the sample collection room, the reclining chair being configured for positioning a subject effective for obtaining a sample; providing flowing water in the sample collection room; providing adjustable lighting in the sample collection room; providing calming sounds in the sample collection room; and providing a calming scent in the sample collection room. Optionally, the method comprises providing a reclining chair in the sample collection room, the reclining chair being configured for positioning a subject effective for obtaining a sample; providing flowing water in the sample collection room; providing adjustable lighting in the sample collection room; providing a calming image in the sample collection room; providing calming sounds in the sample collection room; and providing a calming scent in the sample collection room.

In another embodiment as described herein, a system is provided for obtaining a sample from a subject, comprising a sample collection room in a retail store, the sample collection room comprising one or more of: a warming table, a warming plate, a chair or other furniture for seating comprising a warming plate, a finger warmer, a fingertip warmer, and an air-warmer.

In another embodiment as described herein, a system is provided for obtaining a sample from a subject, comprising a sample collection room in a retail store, the sample collection room comprising a warming table; a reclining chair configured for positioning a subject effective for obtaining a sample; and a calming feature.

In another embodiment as described herein, a system is provided for obtaining a sample from a subject, comprising a sample collection room in a retail store, the sample collection room comprising a chair or other furniture for seating, the chair or other furniture for seating comprising a warming plate or other heating element, the chair or other furniture for seating being configured to warm a body part of the subject, and being configured for positioning a subject effective for obtaining a sample; and a calming feature. Optionally, the warming table is configured to adjust one or more of temperature, height, orientation, distance from a subject, and presence or height of a rest. Optionally, the calming feature is selected from the group of calming features consisting of flowing water, a flower, a plant, a calming scent, a calming sound, a calming image, and a calming color. Optionally, the system further comprises a bathroom adjacent the sample collection room; and a pass-through connecting the sample collection room with the bathroom, wherein the pass-through is configured to allow the transfer of a sample from the bathroom to the sample collection room. Optionally, the sample comprises a urine sample. Optionally, the system comprises one or more of the following: a device for flowing water in the sample collection room; brightness-adjustable lighting in the sample collection room; a calming image; a wall or wall hanging having a calming color; a sound system configured for providing calming sounds in the sample collection room; and a scent source configured to provide a calming scent in the sample collection room. Optionally, the system further comprises a scent source, wherein the scent source is selected from a flower, a candle, an oil, a scented wick, and an aerosol.

In another embodiment, a method is provided for sample collection from a subject, the subject having a body part, the method comprising: obtaining, in a sample collection room, a sample from the body part of the subject; monitoring activity in the sample collection room using at least two motion capture devices positioned to view a field of interest in the sample collection room; and alerting a technician performing the sample collection if a non-conforming action by the technician is detected by at least one of the motion capture devices.

It should be understood that one or more of the following features may be adapted for use with at least one or more of the embodiments described herein. For example, the method may include alerting comprises providing instructions for at least one corrective action to the technician to remedy the non-conforming action. Optionally, alerting further comprises connecting a human by way of a telecommunication link to the technician to guide the technician in at least one corrective action related to the non-conforming action. Optionally, the corrective action comprises changing angulation of a sample collection device relative to horizontal. Optionally, the corrective action comprises using a sample collection device with a different anti-coagulant therein. Optionally, the corrective action comprises applying a warming device to a target tissue for a longer period of time. Optionally, the method comprises attaching at least one motion capture reflective element to the technician. Optionally, the method comprises attaching at least one motion capture reflective element to a garment to be worn by the technician. Optionally, the method comprises providing an accessory to be worn by the technician, wherein the accessory comprises a motion capture reflective material. Optionally, the method comprises providing at least one motion capture glove to be worn by the technician. Optionally, the method comprises providing at least one motion capture finger marker to be worn by the technician. Some embodiments may have the motion capture finger marker only on the nail side of the fingertip of the thumb, index finger, and middle finger of the technician. Optionally, some embodiments may have the motion capture finger marker only on the nail side of the fingertip of the thumb and index finger. Optionally, some embodiments may have the motion capture finger marker only on the nail side of the fingertip one of the thumb, index finger, or middle finger.

Optionally, the method comprises providing at least one motion capture wrist marker to be worn by the technician. Optionally, the method comprises providing at least one motion capture forearm marker to be worn by the technician. Optionally, the sample capture device comprises at least one motion capture marker. Optionally, the method comprises comparing motion captured by the motion capture device with a reference motion stored for that technician when performing the sample collection. Optionally, the method comprises comparing motion captured by the motion capture device with a reference motion for performing the sample collection.

In embodiments, the designated location in the retail store may be placed at the back of the store (i.e., at a location spaced away from the main public entrance, or entrances, to the retail store). In embodiments, the designated location in the retail store may be placed near the front of the store (i.e., at a location close to the main public entrance, or entrances, to the retail store). In embodiments, the designated location in the retail store may be placed along, or near to, a lateral portion of the store (i.e., at a location to the right or to the left of the main public entrance, or entrances, to the retail store). In embodiments, the designated location in the retail store may be placed within the store away from the front, rear, or sides of the retail store.

In embodiments, the designated location in the retail store comprises at least two rooms. In embodiments, the designated location in the retail store comprises at least two rooms, wherein at least one room is configured to serve as a waiting room, and at least one room is configured for obtaining a sample from a subject; in embodiments, the at least one room configured for obtaining a sample from a subject is configured to provide privacy for a subject while a sample is obtained. In embodiments, at least two rooms may be connected by a pass-through (e.g., a passageway) connecting a sample collection room (e.g., a room configured for obtaining samples, such as blood samples) to a bathroom. In embodiments, at least two rooms may be connected by a pass-through (e.g., a passageway) connecting a room configured for obtaining samples to a room configured to serve as a waiting room. In embodiments, at least two rooms may be connected by a pass-through (e.g., a passageway) connecting a room configured for obtaining samples to a room configured to serve as a sample analysis room. In embodiments, such a pass-through or passageway may have a door or hatch having an open configuration and a closed configuration. Such a pass-through may have, for example, a rectangular shape, or a square shape. Such a pass-through may have, for example, a door which may be closed when the pass-through is not in use for transfer of a sample from a patient to sample collection personnel. Such a door may be, for example, a sliding door (e.g., may open and close substantially in the plane of a wall through which the pass-through passes), or may be a hinged door.

In embodiments, a sample collection room may include furniture configured for use during collection of a sample from a subject, such as, e.g., a blood sample, a throat swab, a nasal swab, a cheek swab, a sputum sample, a tear sample, or other sample. For example, a sample collection room may include a chair for use by a subject while a sample is collected. In embodiments, such a chair is a reclining chair, having at least two configurations: an upright configuration (in which a subject seated in the chair assumes a normal seated position with the back in a substantially vertical orientation), and a reclined configuration (in which a subject is leaned backwards when seated in the chair). In embodiments, such a reclining chair provides a leg rest, or foot rest, when in the reclined configuration. In embodiments, such a reclining chair does not present a subject with a leg rest, or foot rest, when in the upright configuration.

In embodiments, the results of the analysis are available within a short period of time. In embodiments, a clinician receives results of the analysis within a short period of time after the sample was obtained. In embodiments, the subject receives results of the analysis within a short period of time after the sample was obtained.

In embodiments, the clinical sample may be a small clinical sample. In embodiments, the clinical sample may be provided for rapid analysis. In embodiments, a small clinical sample may be provided for rapid analysis. In embodiments, the clinical sample may be provided for analysis at the designated location in a retail store, or for analysis in a location adjacent to, or in close proximity to, the designated location in a retail store; such a clinical sample may be a small clinical sample. In embodiments, the clinical sample may be analyzed at the designated location in a retail store, or in a location adjacent to, or in close proximity to, the designated location in a retail store; such analysis may be rapid analysis.

Applicants disclose methods for obtaining a sample from a subject, comprising providing a sample collection room in a retail store; and collecting a sample from a subject. Applicants disclose methods for obtaining a sample from a subject, comprising providing a sample collection room in a retail store; warming a fingertip, or finger, or hand of the subject; collecting a sample from a subject; and obtaining the sample. Applicants disclose methods for obtaining a sample from a subject, comprising providing a sample collection room in a retail store, the sample collection room comprising a warming table; warming a fingertip, or finger, or hand of the subject; and obtaining a sample from the subject. Applicants disclose methods for obtaining a sample from a subject, comprising providing a sample collection room in a retail store, the sample collection room comprising a chair; warming a fingertip, or finger, or hand of the subject; and obtaining a sample from the subject. Applicants disclose methods for obtaining a sample from a subject, comprising providing a sample collection room in a retail store, the sample collection room comprising a chair having a warming plate or other warming element; warming a fingertip, or finger, or hand of the subject; and obtaining a sample from the subject. Applicants disclose methods for obtaining a sample from a subject, comprising providing a sample collection room in a retail store, the sample collection room comprising an air-warmer; warming a fingertip, or finger, or hand of the subject; and obtaining a sample from the subject.

In embodiments, the sample is a small sample. For example, in embodiments of the methods for obtaining a sample from a subject, the sample is obtained from a finger-stick. For example, in embodiments, the sample comprises a few drops, or two drops, or one drop, of blood obtained from a small lancet puncture in the skin of the subject. For example, in embodiments, the sample is a small volume sample of blood, or of urine, or of saliva, or of tears, or other bodily secretion or excretion. For example, in embodiments, the sample is a small sample having a volume of less than about 200 μL, or less than about 150 μL, or less than about 100 μL, or less than about 75 μL, or less than about 50 μL, or less than about 25 μL, or less.

Applicants disclose methods for analyzing a sample obtained from a subject, comprising providing a sample collection room in a retail store; collecting a sample from a subject; and analyzing the sample. Applicants disclose methods for analyzing a sample obtained from a subject, comprising providing a sample collection room in a retail store; warming a fingertip, or finger, or hand of the subject; collecting a sample from a subject; and analyzing the sample. Applicants disclose methods for analyzing a sample obtained from a subject, comprising providing a sample collection room in a retail store, the sample collection room comprising a warming table; warming a fingertip, or finger, or hand of the subject; collecting a sample from a subject; and analyzing the sample. Applicants disclose methods for analyzing a sample obtained from a subject, comprising providing a sample collection room in a retail store, the sample collection room comprising a chair; warming a fingertip, or finger, or hand of the subject; collecting a sample from a subject; and analyzing the sample. Applicants disclose methods for analyzing a sample obtained from a subject, comprising providing a sample collection room in a retail store, the sample collection room comprising a chair having a warming plate or other warming element; warming a fingertip, or finger, or hand of the subject; collecting a sample from a subject; and analyzing the sample. Applicants disclose methods for analyzing a sample obtained from a subject, comprising providing a sample collection room in a retail store, the sample collection room comprising an air-warmer; warming a fingertip, or finger, or hand of the subject; collecting a sample from a subject; and analyzing the sample. Applicants disclose methods for analyzing a sample obtained from a subject, comprising any of the foregoing methods, and providing the subject with a fingertoy, or placing a fingertoy on a finger of a subject following obtaining a sample from the subject. It will be understood that a step of warming a fingertip, or finger, or hand of a subject may comprise warming by use of a warming table, or warming plate, or air-warmer, or other warming device, or a combination thereof. It will be understood that the foregoing methods may be used together, and may be used in any combination.

In embodiments, the sample is analyzed at, or adjacent to, or near to, the sample collection room in the retail store. In embodiments, the sample is analyzed at a location other than at the retail store. In embodiments of the methods for analyzing a sample obtained from a subject, the sample is a small sample. For example, in embodiments, the sample is obtained from a finger-stick. For example, in embodiments, the sample comprises a few drops, or two drops, or one drop, of blood obtained from a small lancet puncture in the skin of the subject. For example, in embodiments, the sample is a small volume sample of blood, or of urine, or of saliva, or of tears, or other bodily secretion or excretion. For example, in embodiments, the sample is a small sample having a volume of less than about 200 μL, or less than about 150 μL, or less than about 100 μL, or less than about 75 μL, or less than about 50 μL, or less than about 25 μL, or less.

Accordingly, Applicants disclose methods for obtaining and analyzing a sample obtained from a subject, comprising: providing a sample collection room in a retail store; collecting a sample from a subject in the sample collection room; and analyzing the sample. Applicants disclose methods for obtaining and analyzing a sample obtained from a subject, comprising: providing a sample collection room in a retail store; collecting a small sample from a subject in the sample collection room; and analyzing the small sample. Applicants disclose methods for obtaining and analyzing a sample obtained from a subject, comprising: providing a sample collection room in a retail store; collecting a sample from a subject in the sample collection room; and analyzing the sample at the retail store location, or adjacent to the retail store location.

In embodiments, a sample collection room may include a comfortable chair or seating location for a subject, e.g., for use by a subject during collection of a sample from the subject. In embodiments, a comfortable chair may be a reclining chair, having an upright configuration and a reclined configuration. In embodiments, this chair may have a medical grade upholstery or covering for hygiene purposes and for ease of cleaning between subjects. In embodiments, a comfortable chair may be a reclining chair having an upright configuration and a plurality of reclined configurations. In embodiments, a sample collection room may include a refrigerator, a cabinet, a sink, storage units, a work surface configured for writing, and other furniture and architectural elements.

In embodiments, a sample collection room may include a sample analysis device or system; such a sample analysis device or system may be an automated sample analysis device or system, or may be a semi-automated sample analysis device or system. In embodiments, a sample collection room may include a computer, computer accessories, communication equipment and capability effective to link a computer, cell phone, or other device with WiFi, internet, cable, or other connections.

Accordingly, Applicants disclose herein systems comprising a room in a retail location, the room being configured for obtaining a clinical sample such as a blood sample. In embodiments, such systems comprise a room in a retail location, the room being configured for obtaining a clinical sample such as a blood sample, the room including one or more of a warming table, a calming feature, a chair, a pass-through to an adjacent bathroom, adjustable lighting, calming scents, and a sound source providing calming sounds. In embodiments, the chair is a reclining chair. In embodiments, the system includes an adjacent bathroom.

In embodiments, the sample collection room may be part of a sample collection center, or wellness center, including a waiting room and a sample collection room. In embodiments, a sample collection room, or wellness center, may include a waiting room, a sample collection room, and a bathroom. In embodiments, a bathroom that is part of a sample collection room, or wellness center, may include a sink (e.g., for handwashing) and a toilet. In embodiments, a bathroom and a sample collection room may be connected via a passageway configured for passing a sample from the bathroom to the sample collection room.

The methods, rooms, and systems disclosed herein are suited for providing subjects with pleasant surroundings, and convenient access to locations where a sample may be obtained for analysis. The methods, rooms, and systems disclosed herein a provide methods and means for obtaining samples from subjects in a pleasant and convenient manner. Thus, the methods, rooms, and systems disclosed herein provide advantages to subjects who wish to provide a sample (e.g., a clinical sample for medical analysis or diagnosis) and provide advantages over the art.

Accordingly, Applicants disclose embodiments of systems for obtaining samples, and systems for analyzing samples, including at least the following.

A room in a retail location configured for obtaining a blood sample including warming table, a calming feature, and a chair, the room being configured for obtaining a clinical sample such as a blood sample. In embodiments, the chair is a reclining chair.

A room in a retail location configured for obtaining a blood sample including a warming table, a calming feature, a pass-through to an adjacent bathroom, and a reclining chair.

A room in a retail location configured for obtaining a blood sample including a warming table, a calming feature, adjustable lighting, a pass-through to an adjacent bathroom, and a reclining chair.

A room in a retail location configured for obtaining a blood sample including a warming table, a calming feature, adjustable lighting, calming scents, a pass-through to an adjacent bathroom, and a reclining chair.

A room in a retail location configured for obtaining a blood sample including a warming table, a calming feature, a calming image, calming sounds, adjustable lighting, calming scents, a pass-through to an adjacent bathroom, and a reclining chair.

A room in a retail location configured for obtaining a blood sample including a warming table, a calming feature, a calming image, calming sounds, calming colors, adjustable lighting, calming scents, a pass-through to an adjacent bathroom, and a reclining chair.

Applicants disclose methods for obtaining samples, and for analyzing samples, including the following methods.

A method for obtaining a sample from a subject, the subject having a body part, comprising: providing a sample collection room in a retail store; and obtaining a sample from the body part of the subject, wherein the obtaining is performed in the sample collection room.

A method for analyzing a sample obtained from a subject, the subject having a body part, comprising: providing a sample collection room in a retail store; obtaining, in the sample collection room, a sample from the body part of the subject; and analyzing the sample at or adjacent to the retail store.

A method for analyzing a sample obtained from a subject, the subject having a body part, comprising: providing a sample collection room in a retail store; obtaining, in the sample collection room, a sample from the body part of the subject; and analyzing the sample at or adjacent to the retail store within a short period of time after obtaining the sample.

A method for analyzing a sample obtained from a subject, the subject having a body part, comprising: providing a sample collection room in a retail store; obtaining, in the sample collection room, a sample from the body part of the subject; transporting the sample to an analysis location; and analyzing the sample at the analysis location within a short period of time after obtaining the sample. In embodiments, obtaining the sample comprises warming the body part of the subject. In embodiments, warming the body part comprises warming with a warming table. In embodiments, warming the body part comprises warming with a warming plate. In embodiments, warming the body part comprises warming with an air-warmer. In embodiments, the body part comprises a fingertip, a finger, a plurality of fingers, a hand, or other body part or body parts.

In embodiments of the foregoing methods, the methods may comprise warming a body part; for example, a body part may be warmed prior to obtaining a sample from the body part. In embodiments, warming of the body part comprises warming with a warming table. In embodiments, warming of the body part comprises warming with a warming plate. In embodiments, warming of the body part comprises warming with a finger warmer. In embodiments, warming of the body part comprises warming with a fingertip warmer. In embodiments, warming of the body part comprises warming with an air-warmer. In embodiments, the body part comprises a fingertip, a finger, a plurality of fingers, a hand, or other body part or body parts.

In embodiments, methods for analyzing a sample comprise analyzing the sample for the presence of an analyte. In embodiments, methods for analyzing a sample comprise analyzing the sample for the presence of a plurality of analytes. In embodiments, methods for analyzing a sample comprise analyzing the sample for the presence of a plurality of analytes, wherein the plurality of analytes includes two or more of a nucleic acid analyte, a peptide (or polypeptide) analyte, an inorganic analyte, a cell or cell type, and other analytes. In embodiments, methods for analyzing a sample comprise analyzing the sample for the presence of a plurality of analytes, wherein the plurality of analytes are detected by methods including two or more of nucleic acid hybridization methods, antibody binding methods, general chemistry methods, cytometric methods, ion-selective electrode methods, and other methods.

In embodiments, methods for analyzing a sample comprise analyzing a sample by automated analysis devices, or by semi-automated analysis devices, or systems comprising such devices. In embodiments, methods for analyzing a sample comprise analyzing a sample at the sample collection center, e.g., in the sample collection room, or in a room adjacent to the sample collection room, or in a room near to the sample collection room. In embodiments, methods for analyzing a sample comprise analyzing the sample in a short period of time. In embodiments, methods for analyzing a sample comprise analyzing a small volume sample. In embodiments, methods for analyzing a sample comprise analyzing a small volume sample in a short period of time.

In embodiments, these methods may further comprise providing a bathroom adjacent the sample collection room; and providing a pass-through connecting the sample collection room with the bathroom, wherein the pass-through is configured to allow the transfer of a sample from the bathroom to the sample collection room. In embodiments, the sample comprises a urine sample.

In embodiments, these methods may further comprise one or more of: providing a reclining chair in the sample collection room, the reclining chair being configured for positioning a subject effective for obtaining a sample; providing a reclining chair in the sample collection room, the reclining chair having a warming plate or other warming element and being configured for positioning a subject effective for obtaining a sample; providing a warming table in the sample collection room, the warming table being effective to warm a body part (e.g., a fingertip, finger, fingers, or hand); providing a warming plate in the sample collection room, the warming plate being effective to warm a body part (e.g., a fingertip, finger, fingers, or hand); providing an air-warmer in the sample collection room, the air-warmer being effective to warm a body part (e.g., a fingertip, finger, fingers, or hand); and providing a calming feature in the sample collection room. In embodiments, the calming feature may be selected from the group of calming features consisting of flowing water, a flower, a plant, a calming scent, a calming sound, a calming image, and a calming color.

In embodiments, these methods may further comprise providing a reclining chair in the sample collection room, the reclining chair being configured for positioning a subject effective for obtaining a sample; providing flowing water in the sample collection room; providing adjustable lighting in the sample collection room; providing calming sounds in the sample collection room; and providing a calming scent in the sample collection room. In embodiments, these methods may further comprise providing a calming image in the sample collection room.

Applicants further provide systems, including the following systems.

A system for obtaining a sample from a subject, comprising a sample collection room in a retail store, the sample collection room comprising a warming table. A system for obtaining a sample from a subject, comprising a sample collection room in a retail store, the sample collection room comprising a warming plate. A system for obtaining a sample from a subject, comprising a sample collection room in a retail store, the sample collection room comprising a chair, couch, bench, or other piece of furniture for seating, wherein the chair, couch, bench, or other piece of furniture has a warming plate or warming element in an armrest of the chair, couch, bench, or other piece of furniture. A system for obtaining a sample from a subject, comprising a sample collection room in a retail store, the sample collection room comprising a finger warmer. A system for obtaining a sample from a subject, comprising a sample collection room in a retail store, the sample collection room comprising a fingertip warmer. A system for obtaining a sample from a subject, comprising a sample collection room in a retail store, the sample collection room comprising an air-warmer.

A system for obtaining a sample from a subject, comprising a sample collection room in a retail store, the sample collection room comprising a warming table; a reclining chair (or other piece of furniture for seating) configured for positioning a subject effective for obtaining a sample; and a calming feature. In embodiments, a warming table may be configured to adjust one or more of temperature, height, longitudinal orientation, lateral orientation, distance from a subject, and presence or height of a rest. In embodiments, a chair (or other piece of furniture for seating) may include a warming plate or warming element in an armrest. In embodiments, a calming feature may be selected from the group of calming features consisting of flowing water, a flower, a plant, a calming scent, a calming sound, a calming image, and a calming color.

In embodiments, the system may further comprise a bathroom adjacent the sample collection room; and a pass-through connecting the sample collection room with the bathroom, wherein the pass-through is configured to allow the transfer of a sample from the bathroom to the sample collection room. In embodiments, the sample comprises a urine sample.

In embodiments, such systems may further comprise one or more of: flowing water in the sample collection room; adjustable lighting in the sample collection room; a calming image; a wall or wall hanging having a calming color; a sound system configured for providing calming sounds in the sample collection room; and a scent source configured to provide a calming scent in the sample collection room. In embodiments, a scent source may be selected from a flower, a candle, an oil, a scented wick, and an aerosol.

In embodiments, systems as disclosed herein may further include a warming plate, or a finger warmer, or a fingertip warmer, or an air-warmer, or combination thereof. In embodiments, systems as disclosed herein may further include a fingertoy. It will be understood that systems as disclosed herein may include combinations of some or all of the elements disclosed herein.

In yet another embodiment, a method is provided for sample collection from a subject, the method comprising: obtaining, in a sample collection room, a sample from the body part of the subject; monitoring activity in the sample collection room using at least two motion capture devices positioned to view a field of interest in the sample collection room; and alerting a technician performing the sample collection if a non-conforming action by the technician is detected by at least one of the motion capture devices. It should be understood that, in embodiments, any of the sample collection oversight technique(s) may be applied to any of the sample collection rooms or POS sites discussed elsewhere herein. In embodiments, the oversight herein can be open-loop without direct real-time feedback to the technician performing the collection, delayed closed-loop with feedback given at a different time (not real time) to the technician, or closed-loop real-time with feedback given to the technician during the collection process. In some embodiments, the feedback may be given through an ear piece, headset, headphone, heads-up display, eye piece, glasses, or other communication system that is primarily only observable by the technician and not the subject from whom sample is being collected.

It should be understood that any of the embodiments herein may include one or more the following features. For example, the method of claim 1 wherein alerting comprises providing instructions for at least one corrective action to the technician to remedy the non-conforming action. Optionally, alerting further comprises connecting a human by way of a telecommunication link to the technician to guide the technician in at least one corrective action related to the non-conforming action. Optionally, the corrective action comprises changing angulation of a sample collection device relative to horizontal. Optionally, the corrective action comprises using a sample collection device with a different anti-coagulant therein. Optionally, the corrective action comprises applying a warming device to a target tissue for a longer period of time. Optionally, the method further comprises attaching at least one motion capture reflective element to the technician. Optionally, the method further comprises attaching at least one motion capture reflective element to a garment to be worn by the technician. Optionally, the method further comprises providing an accessory to be worn by the technician, wherein the accessory comprises a motion capture reflective material. Optionally, the method further comprises providing at least one motion capture glove to be worn by the technician. Optionally, the method further comprises providing at least one motion capture finger marker to be worn by the technician. Optionally, at least one motion capture wrist marker is to be worn by the technician. Optionally, at least one motion capture forearm marker to be worn by the technician. Optionally, the sample capture device comprises at least one motion capture marker. Optionally, a sample collection device used for collection comprises at least one motion capture marker. Optionally, the method further comprises comparing motion captured by at least one of the motion capture devices with a reference motion stored for that technician when performing the sample collection. Optionally, the method further comprises comparing motion captured by at least one of the motion capture devices with a reference motion for performing the sample collection. Optionally, a method is provided for sample collection motion tracking verification. Optionally, a device is provided for sample collection motion tracking verification. Optionally, a sample collection motion tracking system is provided. Optionally, a sample collection motion tracking method is provided. Optionally, a sample collection quality inspection system is provided. Optionally, a sample collection quality inspection method is provided. Optionally, the method comprises at least one technical feature from any of the prior features. Optionally, the method comprises at least any two technical features from any of the prior features. Optionally, the device comprises at least one technical feature from any of the prior features. Optionally, the device comprises at least any two technical features from any of the prior features. Optionally, the system comprises at least one technical feature from any of the prior features. Optionally, the system comprises at least any two technical features from any of the prior features.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1A shows a plan view of one embodiment of a sample collection room within a retail location, having a waiting room, a sample collection room, and a bathroom. Exemplary positioning of furniture, work surfaces, and doors are indicated in the figure.

FIG. 1B shows a plan view of another embodiment of a sample collection room.

FIG. 2 shows an example of a warming table having features as disclosed herein.

FIG. 3 shows an example of a warming table having features as disclosed herein, including an arm rest.

FIG. 4 shows a finger warmer wrapped around a finger of a subject.

FIG. 5 shows a schematic view of one embodiment of a system for motion capture.

FIG. 6 shows one embodiment of a sample collection process.

### DETAILED DESCRIPTION

Description and disclosure of examples of obtaining samples and of sample analysis methods, devices, and systems, including automated analysis devices, semi-automated analysis devices, and systems comprising such devices, which may be used with the methods and systems disclosed herein may be found, for example, in U.S. Pat. No. 8,088,593; U.S. Pat. No. 8,380,541; U.S. patent application Ser. No. 13/769,820, filed Feb. 18, 2013; PCT/US2012/57155, filed Sep. 25, 2012; U.S. Application Ser. No. 61/673,245, filed Sep. 26, 2011; U.S. Pat. No. 8,088,593; in U.S. Pat. No. 8,380,541; in International Patent Application PCT/US2013/052141, filed Jul. 25, 2013; U.S. patent application Ser. No. 13/244,947 filed Sep. 26, 2011; and in U.S. application Ser. No. 13/244,946, filed Sep. 26, 2011; the disclosures of all which patents and patent applications are hereby incorporated by reference in their entireties.

It is to be understood that both the foregoing general description and the following detailed description are exemplary and explanatory only and are not restrictive of the invention, as claimed. It may be noted that, as used in the specification and the appended claims, the singular forms “a”, “an” and “the” include plural referents unless the context clearly dictates otherwise. Thus, for example, reference to “a material” may include mixtures of materials, reference to “a compound” may include multiple compounds, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for a sample collection unit, this means that the sample collection unit may or may not be present, and, thus, the description includes both structures wherein a device possesses the sample collection unit and structures wherein sample collection unit is not present.

As used herein, the terms “substantial” means more than a minimal or insignificant amount; and “substantially” means more than a minimally or insignificantly. Thus, for example, the phrase “substantially different”, as used herein, denotes a sufficiently high degree of difference between two numeric values such that one of skill in the art would consider the difference between the two values to be of statistical significance within the context of the characteristic measured by the values. Thus, the difference between two values that are substantially different from each other is typically greater than about 10%, and may be greater than about 20%, preferably greater than about 30%, preferably greater than about 40%, preferably greater than about 50% as a function of the reference value or comparator value.

As used herein, the term “point of service location” (POS) may include locations where a subject may receive a service (e.g. testing, monitoring, treatment, diagnosis, guidance, sample collection, ID verification, medical services, non-medical services, etc.), and may include, without limitation, a subject's home, a subject's business, the location of a healthcare provider (e.g., doctor), hospitals, emergency rooms, operating rooms, clinics, health care professionals' offices, laboratories, retailers [e.g. pharmacies (e.g., retail pharmacy, clinical pharmacy, hospital pharmacy), drugstores, supermarkets, grocers, etc.], transportation vehicles (e.g. car, boat, truck, bus, airplane, motorcycle, ambulance, mobile unit, fire engine/truck, emergency vehicle, law enforcement vehicle, police car, or other vehicle configured to transport a subject from one point to another, etc.), traveling medical care units, mobile units, schools, day-care centers, security screening locations, combat locations, health assisted living residences, government offices, office buildings, tents, bodily fluid sample acquisition sites (e.g. blood collection centers), sites at or near an entrance to a location that a subject may wish to access, sites on or near a device that a subject may wish to access (e.g., the location of a computer if the subject wishes to access the computer), a location where a sample processing device receives a sample, or any other point of service location described elsewhere herein.

As used herein, a “sample”, or “biological sample”, or “clinical sample” refers to a sample of fluid, tissue, secretion, or excretion obtained from a subject. A clinical sample may be a sample of blood, serum, plasma, saliva, sputum, urine, gastric fluid, digestive fluid, tears, sweat, stool, semen, vaginal fluid, interstitial fluid, fluid derived from tumorous tissue, ocular fluids, mucus, earwax, oil, glandular secretions, spinal fluid, skin, cerebrospinal fluid from within the skull, tissue, fluid or material from a nasal swab, a throat swab, a cheek swab, or nasopharyngeal wash, biopsy fluid or material, placental fluid, amniotic fluid, cord blood, lymphatic fluids, cavity fluids, pus, microbiota obtained from a subject, meconium, breast milk, or other secretion or excretion. A sample may be a breath sample, a hair sample, a fingernail sample, or other sample.

Biological samples may include nasopharyngeal wash, or other fluid obtained by washing a body cavity or surface of a subject, or by washing a swab following application of the swab to a body cavity or surface of a subject. Nasal swabs, throat swabs, stool samples, hair, finger nail, ear wax, breath, and other solid, semi-solid, or gaseous samples may be processed in an extraction buffer, e.g., for a fixed or variable amount of time, prior to their analysis. The extraction buffer or an aliquot thereof may then be processed similarly to other fluid samples if desired. Examples of tissue samples of the subject may include but are not limited to, connective tissue, muscle tissue, nervous tissue, epithelial tissue, cartilage, cancerous sample, or bone. The sample may be obtained from a human or animal. The sample may be obtained from a vertebrate, e.g., a bird, fish, or mammal, such as a rat, a mouse, a pig, an ape, another primate (including humans), a farm animal, a sport animal, or a pet. The sample may be obtained from a living or dead subject. The sample may be obtained fresh from a subject or may have undergone some form of pre-processing, storage, or transport.

Thus, as used herein, a “sample” may be but is not limited to a blood sample, or a portion of a blood sample, may be of any suitable size or volume, and is preferably of small size or volume. In some embodiments of the assays and methods disclosed herein, measurements may be made using a small volume blood sample, or no more than a small volume portion of a blood sample, where a small volume comprises no more than about 5 mL; or comprises no more than about 3 mL; or comprises no more than about 2 mL; or comprises no more than about 1 mL; or comprises no more than about 500 μL; or comprises no more than about 250 μL; or comprises no more than about 100 μL; or comprises no more than about 75 μL; or comprises no more than about 50 μL; or comprises no more than about 35 μL; or comprises no more than about 25 μL; or comprises no more than about 20 μL; or comprises no more than about 15 μL; or comprises no more than about 10 μL; or comprises no more than about 8 μL; or comprises no more than about 6 μL; or comprises no more than about 5 μL; or comprises no more than about 4 μL; or comprises no more than about 3 μL; or comprises no more than about 2 μL; or comprises no more than about 1 μL; or comprises no more than about 0.8 μL; or comprises no more than about 0.5 μL; or comprises no more than about 0.3 μL; or comprises no more than about 0.2 μL; or comprises no more than about 0.1 μL; or comprises no more than about 0.05 μL; or comprises no more than about 0.01 μL.

As used herein, a “small volume” refers to a volume of less than about 1 mL, or less than about 500 μL, or less than about 250 μL, or less than 150 μL, or less than about 100 μL, or less than about 50 μL, or less than about 25 μL, or less. In particular embodiments, a small volume, such as a “finger-stick” volume, may comprise less than about 250 μL, and typically comprises less than 150 μL, or less than about 100 μL, or less than about 50 μL, or less than about 25 μL, or less.

As used herein, a “short period of time” refers to a period of time of about 5 hours or less, or about 4 hours or less, or about 3 hours or less, or about 2 hours or less, or about 1 hour or less, or about 50 minutes or less, or about 40 minutes or less, or about 30 minutes or less, or about 20 minutes or less, or about 10 minutes or less, or about 5 minutes or less. A short period of time may be determined with respect to an initial time; the initial time may be the time at which a sample analysis began; the initial time may be the time at which a sample is inserted into a device for the analysis of the sample; the initial time may be the time at which a sample was obtained from a subject.

It should be understood that embodiments in this disclosure may be adapted to have one or more of the features described below.

Systems, methods, and devices for obtaining samples are provided. Such samples are provided for analysis in order to detect a diseases or condition of a subject. Such samples may be used, for example, to detect the presence of markers indicative of one or more of a plurality of infectious agents in a single clinical sample, or in a plurality of aliquots of a single clinical sample. The systems, methods, and devices disclosed herein may be point-of-service systems, methods, and devices, configured for use at a point-of-service location, where a point-of-service location may be a location at which a sample is obtained from a subject. In the embodiments disclosed herein, a sample is obtained from a subject at a retail location. In particular, a sample is obtained from a subject at a designated sample collection location within a retail location.

In embodiments, the sample is a small-volume sample. In embodiments, the sample is used to test for a plurality of markers, or a plurality of diseases; such testing may include nucleic acid testing (e.g., testing for DNA or RNA sequences present in the sample), amino acid testing (e.g., using antibodies or binding proteins), general chemistry testing, cytometric testing (e.g., producing and analyzing images of cells in a sample, or using other optical and microscopic techniques), and other testing. In embodiments, the sample is collected at the retail location, and is analyzed at another location. In embodiments, the analysis of the small-volume clinical sample is completed in a short period of time. In embodiments, the sample is collected at the retail location, and is analyzed in a device at the retail location. In embodiments, the analysis of the small-volume clinical sample is completed in a short period of time.

Methods for obtaining a sample from a subject include providing a sample collection room within a retail store; obtaining a sample from the subject; and analyzing the sample. Samples may be collected in a sample collection room adjacent a waiting room; a bathroom may be provided with a pass-through to the sample collection room. A sample may be sent to a remote location for analysis. A sample may be analyzed at, or adjacent to, the sample collection location. A sample collection room may house a sample analysis device or sample analysis system, and thus also serve as a sample analysis room. A sample may be a small sample, e.g., a small blood sample may be taken from a single finger-stick, or two or a few finger-sticks. A sample may be analyzed in a short period of time, e.g., in less than five hours, or less than four hours.

Methods for obtaining a sample from a subject include providing a sample collection room within a retail store, and obtaining a sample from the subject.

Methods for analyzing a sample obtained from a subject include providing a sample collection room within a retail store; obtaining a sample from the subject; and analyzing the sample. In embodiments of methods for analyzing a sample obtained from a subject the sample is analyzed at the retail location. A sample collection room may be adjacent a waiting room; a bathroom may be provided; a pass-through from bathroom to sample collection room may be provided.

A warming table may be provided in a sample collection room which has an integrated passthrough window specially designed to transfer urine into the blood collection area and into sample analysis devices or systems.

In embodiments of methods for obtaining a blood sample from a subject, blood may be obtained from a small skin puncture. Such a small skin puncture may be, e.g., a small skin puncture on a finger, such as on a finger tip; a small skin puncture on a toe, such as on or near a tip of a toe; a small skin puncture on a foot, such as on or near the heel; or other small skin puncture. A small skin puncture is on a finger may be termed a “finger-stick”; the term finger-stick may also be used to describe the volume of a sample, i.e., a finger-stick volume is one, two, or a few drops (e.g., about 50 μL to about 300 μL, or about 75 μL to about 250 μL, or about 80 μL to about 150 μL).

In embodiments, a sample collection room includes a location for a practitioner (e.g., a sample collection technician) to sit and obtain a bodily fluid or other sample from the patient.

In embodiments, a work surface such as a table is provided so that a patient has a surface on which they can position the target site on the subject to allow for sampling.

In embodiments, the work surface may have one or more thermal controlled sites wherein the temperature of the target site on the patient may be brought to a desired temperature. The temperature may be adjustable. By way of example and not limitation, embodiments may heat a finger or other target site to improve blood flow and thus blood yield from a finger-stick. For example, the table may have thermal control areas to increase blood flow to the target area and thus increase the speed with which sufficient blood or other bodily fluid can be drawn from the subject. The heating is used to bring the target tissue to between about 40° C. to about 50° C. In embodiments, the heating brings target tissue to within a temperature range of between about 40° C. to about 44° C. In embodiments, the heating brings target tissue to within a temperature range of between about 41° C. to about 43° C. In embodiments, the heating brings target tissue to a temperature of about 42° C. In embodiments, the heating brings target tissue to within a temperature range of between about 44° C. to about 47° C. In one embodiment, the temperature is sufficient to increase blood flow to yield 120 uL of sample. In one embodiment, the temperature is sufficient to increase blood flow to yield 130 uL of sample. In one embodiment, the temperature is sufficient to increase blood flow to yield 140 uL of sample. In one embodiment, the temperature is sufficient to increase blood flow to yield 150 uL of sample. Optionally, the thermal controlled site is a shaped surface is contoured to match that of the target site on a patient.

In embodiments, the thermal controlled site comprises a hard surface that is easily cleaned, and that may be sterilized. Such a surface may be or include, for example, plastic, glass, hard rubber, acrylic, polymer, and other materials. In embodiments, the thermal controlled site comprises a soft or padded surface that is easily cleaned, and that may be sterilized. Such a surface may be or include, for example, cloth, rubberized cloth, soft rubber, and other materials.

In embodiments, such a temperature-controlled work surface is provided as part of a table, which may be termed a warming table. Accordingly, a sample collection room may include a warming table. A warming table may be configured to warm a subject's hand, foot, or other body part. The warming table may be tiltable; and its height may be adjustable. A warming table may be configured for use with the left hand, or right hand, of a subject. A warming table may be configured for use with the left foot, or right foot, of a subject. For example, a warming table may be configured for placement near a chair (such as a reclining chair) on either side of a subject seated in the chair.

It will be understood that a warming table may be any fixture or piece of furniture which provides a temperature-controlled surface on which a subject may place a part of their body (e.g., hand, arm, foot, or other part) for obtaining a sample. Thus, a warming table may provide such a temperature-controlled surface configured to receive a part of a subject's body; may provide such a temperature-controlled surface and also provide storage, e.g., for supplies; may provide such a temperature-controlled surface and also provide guides for placement of a hand, arm, foot, or other part of a subject's body; may provide such a temperature-controlled surface and also provide lighting, e.g., for illuminating the subject or a part of a subject's body; a waste receptacle; and other features and elements, and combinations thereof. Supply storage may be provided by drawers, shelves, bins, slots, racks, or other features. A waste receptacle may be configured to receive any waste, may be configured to receive biohazard waste (e.g., waste containing bodily fluids or tissues); or both.

In embodiments, a warming table is adjustable, effective to adjust the temperature of a surface of the warming table (e.g., a surface in contact with a body part of a subject from whom a sample is, or is to be, obtained). In embodiments, a warming table is adjustable, effective to adjust the height of the warming table, including effective to adjust the height of a warming surface. In embodiments, a warming table is adjustable, effective to adjust the orientation, or the tilt, of the warming table. In embodiments, a warming table is adjustable, effective to adjust the distance of the warming table (or a surface thereof) from a subject. In embodiments, a warming table is adjustable, effective to adjust the presence of, position of, and orientation of, a rest, such as a hand or arm rest, on or near the warming table.

In embodiments, a warming table is configured to place the hand or other body part from which a blood sample is to be collected at an optimal height and orientation for facilitating the flow of gravity in the finger as blood droplets form. A warming table may include a rest, such as an arm rest, or hand rest, or other rest which provides an additional surface for contacting a subject; such a surface may make positioning of a hand, or arm, or foot, or leg, other body part more comfortable for the subject, more convenient for the sample collection technician, or both. Such a rest may be configured for placement in more than one position or orientation. Such a rest may be retractable or otherwise configured to be placed or stored out of the way of a subject or sample collection technician when not needed. A warming table may be designed to accommodate the seating or placement of a sample collection technician near to a subject. A warming table, or surface thereof, may be adjustable, or may include an adjustable surface, which may be placed or oriented for the comfort and convenience of a subject, a sample collection technician, or both. For example, a warming table may include a sliding surface which may be extended or retracted as needed to accommodate a subject or a sample collection technician; may include a tiltable surface which may be oriented as needed to accommodate a subject or a sample collection technician; may include a surface which may be raised or lowered as needed to accommodate a subject or a sample collection technician; or be otherwise adjustable.

Accordingly, one or more of the lateral position, the height, and the orientation (e.g., tilt or camber) of a warming table, or a surface thereof, may be adjustable. For example, the height of an upper surface, or portion thereof, of a warming table may be adjustable, e.g., to accommodate the comfort or convenience of the subject, of the sample collection technician, or both. Similarly, the orientation of an upper surface, or portion thereof, of a warming table may be adjustable, e.g., to accommodate the comfort or convenience of the subject, of the sample collection technician, or both. In embodiments, the upper surface of a warming table, or a portion thereof, may be configured for easy cleaning and for maintaining sterility during sample collection. The upper surface of a warming table may be padded, or include a padded portion. The upper surface of a warming table may be water-proof, or water-resistant, or include a water-proof, or water-resistant portion.

In embodiments, the height (as measured from the floor) of the upper surface of a warming table configured to receive a part of a subject's body (e.g., during sample collection) may be between about 10 inches and about 40 inches; or between about 15 inches and 30 inches; or between about 16 inches and 28 inches; or between about 20 inches and about 24 inches in height. In embodiments, the length of a warming table may be between about 20 inches and about 60 inches; or between about 24 inches and 54 inches; or between about 20 inches and 50 inches; or between about 35 inches and about 45 inches in length. In embodiments, the width of a warming table may be between about 8 inches and about 30 inches; or between about 10 inches and 24 inches; or between about 12 inches and 18 inches; or between about 11 inches and about 15 inches in width. In embodiments, a warming table may have a length of about 38 to 42 inches, a width of 12 to 15 inches, and a height of about 19 to about 23 inches.

In embodiments, the orientation of the upper surface of a warming table configured to receive a part of a subject's body (e.g., during sample collection) may be substantially parallel to the floor of the sample collection room.

A subject may sit or lie next to a warming table during sample collection. When seated or lying next to a warming table, the subject presents a subject orientation axis extending from the subject's head towards the subject's feet. The orientation of the upper surface of a warming table configured to receive a part of a subject's body (e.g., during sample collection) may be adjustable; in embodiments, such orientation may be adjustable along an axis substantially parallel to the subject orientation axis; or may be adjustable along an axis substantially perpendicular to the subject orientation axis. In embodiments, the orientation of the upper surface of a warming table may be adjustable along two axes (e.g., along an axis substantially parallel to, and along an axis substantially perpendicular to, the subject orientation axis). It will be understood that the orientation of an upper surface of a warming table may be adjustable along other axes, or in other ways, as well as or instead of along these described axes. In embodiments, the orientation of the upper surface of a warming table may be adjustable between angles between about 0° to about 90°, or between about 0° to about 45°, or between about 0° to about 30°, or other ranges (where 0° indicates level, i.e., substantially parallel to the floor).

It will be understood that adjustment of the height, or orientation, or both, of an upper surface of a warming table may comprise adjustment of a portion of the warming table other than the upper surface; for example, adjustment of the height or orientation of an upper surface of a warming table may be effected by adjusting a leg, or legs, or the warming table, or by adjusting the surface (e.g., the floor or a portion of the floor, or other surface on the floor) on which the warming table is placed; by adjusting an upper portion of the table; by adjusting a lower portion of the table; by adjusting an intermediate portion of the table; or combinations thereof.

A warming table may be used to warm the fingers of a subject prior to puncturing a finger to obtain a blood sample. The thermal regulation of the warming table may be used in conjunction with a finger warmer (a portable device for placement on or around a finger, fingers, or hand of a subject. The thermal regulation of the warming table may be used in place of a finger warmer. In embodiments, a warming table may include a built-in waste receptacle. In embodiments, such a built-in waste receptacle may be, or may include, a biohazard bin.

A sample collection room may include a chair. In embodiments, the chair may be a reclining chair. The chair may be configured for ease of placement of an arm or hand of a subject effective to place the hand of the subject on a warming table, e.g., in proper placement on a warming table for effective warming of the hand prior to obtaining a blood sample. Where a foot of the subject is to be used for obtaining a sample, the chair may be configured for ease of placement of a leg or foot of a subject effective to place the foot of the subject on a warming table, e.g., in proper placement on a warming table for effective warming of the hand prior to obtaining a blood sample. A leg or foot may be preferred, for example, for pediatric patients, or amputees, or patients with impaired blood flow to a hand, or other subjects. A booster, pillow, insert, or other item or feature may be used to position a pediatric or other patient in proper position for obtaining the sample.

Such a chair may be placed adjacent a warming table, effective that a subject may comfortably place their hand, or other body part to be used for sample collection, on the warming table. In embodiments, the chair and the warming table are configured for use together. In embodiments, the chair is connected to the warming table. In embodiments, the chair and the warming table form a single unit. In embodiments, the chair and the warming table form a single unit after connection (e.g., latching, locking, or otherwise securing) of the chair and warming table together. In embodiments, the chair and the warming table are modified to become a single unit (e.g., by screws, nails, clamps, or other permanent or semi-permanent fastening). In embodiments, the chair and the warming table are built as a single unit.

In embodiments, calming features may be selected from flowing water, flowers, plants, scents, sounds, colors, images, and other features and elements which may reduce any anxiety felt by a subject prior to, during, or after obtaining a sample. A flower may be a cut flower, a flower growing as part of a plant, an artificial flower, or an image of a flower.

A sample collection room may include a calming feature. For example, a calming feature may include flowing water (e.g., a wall fixture with flowing water, such as water flowing down from an upper location along a face to a lower collecting trough or frame; a fountain; a rock or rocks over which water flows; an artificial stream; and other flowing water features). A calming feature, including a water feature, may also include a plant, or plants, including ferns, bamboo, moss, flowering plants (e.g., orchids, roses, and other plants), bonsai trees and plants, and other plants. A water feature may produce calming sounds.

A calming feature may include calming sounds in addition to, or exclusive of, a water feature; for example, speakers may provide recorded or synthesized music or sounds. Such sounds may include white noise sounds, beach sounds, forest sounds, bird sounds, traffic sounds, and other sounds, and combinations thereof.

A calming feature may include calming scents, which may be provided by candles present in the sample collection room, or may be provided by wicks, pads, tapes, oils, aerosols, or other means present in the sample collection room or connected to vents and air-channels connected to the sample collection room. Such calming scents may include perfumes, aromas, and other scents and scented materials, whether natural, artificial, or combinations thereof. Calming scents include, e.g., vanilla, rose scent, lavender, coconut, marjoram, chamomile, lilac, citrus scents, and others. Such scents may be provided by flowers (e.g., rose, jasmine, geraniums, and others) or plants (e.g., sage, mint, rosemary, and others) present in the sample collection room or placed in or near sources of airflow into the sample collection room.

In embodiments, the walls, furniture, fixtures, and other elements of a sample collection room may be white or off-white. In embodiments, the walls, furniture, fixtures, and other elements of a sample collection room may be colored in calming colors, e.g. light pastel colors, including greens, blues, yellows, and other light colors. In embodiments, black, red, purple, and other colors may be avoided.

A sample collection room may have adjustable lighting, so that the light intensity may be adjusted from dim to bright, and other levels in between, effective to provide a calming or reassuring effect on a subject.

A sample collection room may include a kiosk or work station configured to provide a sample collection technician with the supplies and equipment needed for sample collection in a location at, adjacent, or near to the collection location. For example, such a kiosk or work station may include, or be placed near to, a table configured for placing a limb of a subject in position for obtaining a sample. In embodiments, such a table may be a warming table.

A kiosk or work station, as disclosed in U.S. Patent Application 61/852,484, may include a user accommodation section adapted to locate a user in at least one position that enables an interaction between the station and the user; a user interface adapted to permit a user to input data relevant to the user; wherein the at least one position enables the user to position a portion of their body onto a work surface for obtaining patient service. Such a workstation may include a secured, temperature controlled sample storage location that is configured to be accessible through a first opening; and a lockable cover for securing the user interface; in embodiments, the lockable cover may not secure the sample storage location. In embodiments, a secured, temperature controlled sample storage location may be configured to be accessible through a first opening and a second opening, wherein a key, code, or structure to open one opening does not open the other opening.

In embodiments, a sample may be sent to a remote location for analysis. A sample may be analyzed at, or adjacent to, the sample collection location. A sample collection room may house a sample analysis device or sample analysis system, and thus also serve as a sample analysis room. A sample may be a small sample, e.g., a small blood sample may be taken from a single finger-stick, or two or a few finger-sticks. A sample may be analyzed in a short period of time, e.g., in less than five hours, or less than four hours.

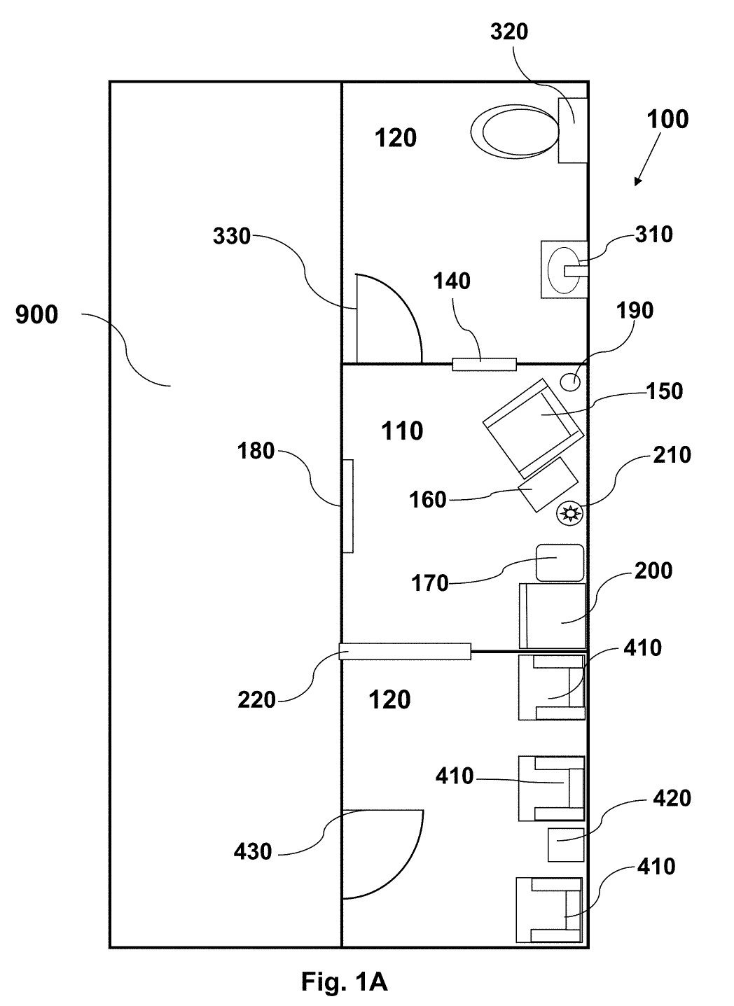

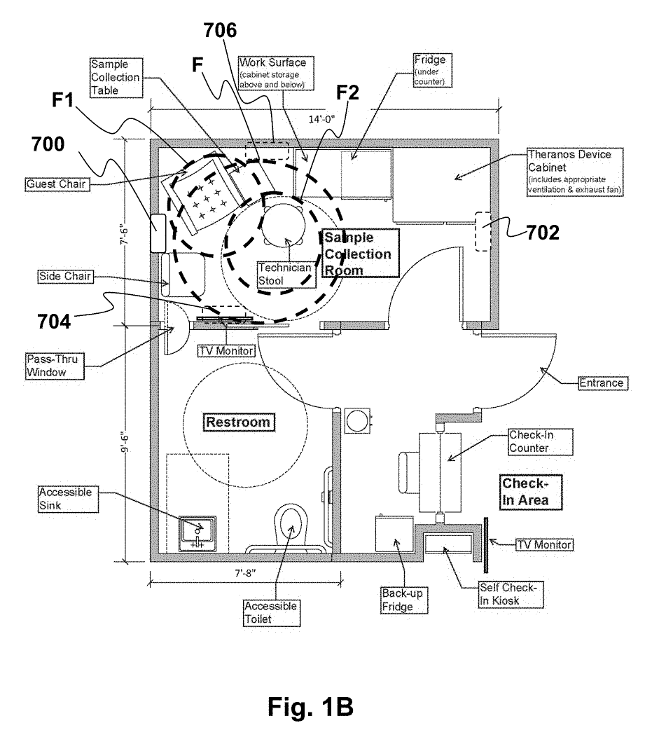

FIG. 1A shows an exemplary system comprising a sample collection center in a retail location, including a waiting room, a sample collection room, a bathroom, a pass-through connecting the bathroom with the sample collection room, and including an (optional) sample analysis device located in the sample collection room. As shown in FIG. 1A, a system may be a sample collection center 100 in a retail store (a portion of the store is indicated by 900). The sample collection center 100 includes a sample collection room 110, a waiting room 120, and a bathroom 130; sample collection room 110 and the bathroom 130 are connected by a pass-through 140, which may be used, e.g., to pass a urine or other sample from the bathroom 130 to the sample collection room 110. The sample collection room 110 includes a reclining chair 150; a warming table 160; an (optional) sample analysis device 170 for analyzing the sample at the sample collection center; a wall-hanging with flowing water 180; an adjustable lamp 190; a refrigerator 200; and a plant 210. It will be understood that air flowing into the sample collection room 110 may be scented, and that soothing music or sounds may be provided in the sample collection room 110. Access to the sample collection room 110 from the waiting room 120 may be by door 220 (shown as a sliding door). Access to the waiting room 120 from retail store 900 may be via door 430. A subject may enter the bathroom 130 from the sample collection room 110 via door 330. Bathroom 130 includes a sink 310 and a toilet 320. Chairs 410 are placed in the waiting room 120 for the convenience of subjects waiting to have samples taken; a table 420 may provide space for informative literature or for light reading material for waiting subjects.

FIG. 1B shows a still further embodiment wherein the waiting area is configured as a check-in area, with the understanding that the subjects may wait and/or shop in the retail or other area coupled to the rooms shown in FIG. 1B. FIG. 1B also shows how the image and/or motion capture sensors 700, 702 (shown in phantom), 704 (shown in phantom), and/or 706 (shown in phantom) may be mounted in a sample collection room to observe the sample collection process. Optionally, some embodiments may include the image and/or motion capture sensor 700 in one or more other rooms such as the check-in area to monitor activity there in terms of the check-in of subjects or the adjustment to appointment times based on the number of subjects in the check-in queue. The system may also calculate wait time based on imaging and display wait time information based on when a subject lined-up for the queue and when they exited the sample collection room and then the check-in area.

FIG. 1B also shows that in some embodiments, the image and/or motion capture sensors 700, 702 (shown in phantom), 704 (shown in phantom), and/or 706 (shown in phantom) may be monitoring one field of view F. Some embodiments may only use one of the foregoing, while others may use two or more of the foregoing sensors and using them in single or multiple combinations to assist in visualizing the perform area of the collection process. Optionally, some may have at least one of the sensors monitoring another field of view F1. Optionally, some may have at least one of the sensors monitoring another field of view F2. Optionally, some may have at least one sensor monitoring field of view F, at least another sensor monitoring F1, and at least another sensor monitoring F2. Optionally, some may use one or more sensors to monitor only F and F1. Optionally, some may use one or more sensors to monitor only F and F2. Optionally, some may use one or more sensors to monitor only F1 and F2. Although FIG. 1B shows mounting of the sensors 700 to 706 on the walls, it should also be understood that other mounting locations such as overhead on the ceiling, underneath in the floor area, and/or on an articulated swing arm (wall mounted or floor mounted) to allow for positioning of the sensor(s) in the desired orientation. Some of the sensors may also tailor their area of interest to certain sub-areas within the observable field so that motions outside the sub-area does not trigger the oversight process. A human (local or at a remote location) may also be alerted by wireless or other electronic communication methods to observe the process if certain parameters are triggered.

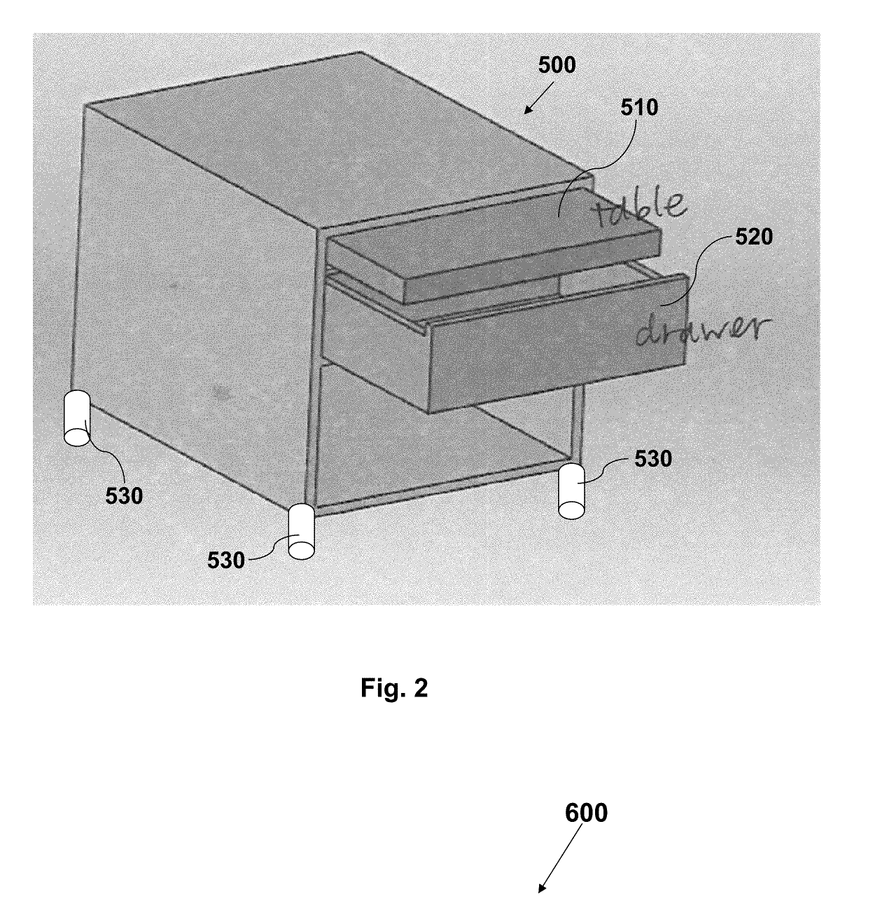

FIG. 2 shows an example of a warming table having a warming surface. The warming table 500 has a warming surface 510 which may be placed in any of several desired locations; as shown, warming surface 510 may slide in and out of a slot. Warming surface 510 also provides a support surface for a body part of a subject, such as an arm or hand, for use during sample collection. Part or all of warming surface 510 may be warmed. Warming table 500 has storage drawers 520 as shown, and adjustable legs 530. The height of adjustable legs 530 may be altered independently of one another, to provide tilt; or may be adjusted together to provide a level surface, as desired.

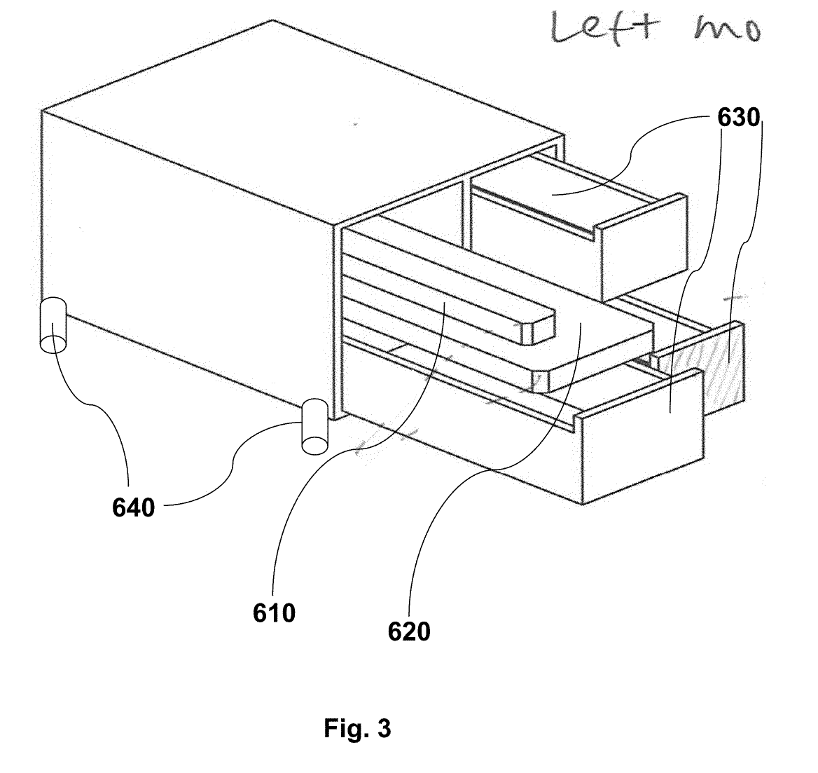

FIG. 3 shows an example of a warming table including an arm rest as well as a warming surface. Warming table 600 has an extendable arm rest 610 which may be disposed adjacent warming surface 620 for the comfort and convenience of a subject and for more effective sample collection. Warming table 600 has storage drawers 630 as shown, and adjustable legs 640. The height of adjustable legs 640 may be altered independently of one another, to provide tilt; or may be adjusted together to provide a level surface, as desired.

Applicants further disclose a warming plate, which includes a warming element and a surface for contacting a finger, or fingers, or a hand of a subject. In embodiments, the upper surface of a warming plate, or a portion thereof, may be configured for easy cleaning and for maintaining sterility during sample collection. The upper surface of a warming plate may be padded, or include a padded portion. The upper surface of a warming plate may be water-proof, or water-resistant, or include a water-proof, or water-resistant portion.

A warming plate as disclosed herein may be a flat plate, suitable for resting on a table, or desk, or shelf, or other surface. A warming plate as disclosed herein may be a flat plate embedded in a table, or desk, or shelf, or other surface. A warming plate as disclosed herein may be a flat plate embedded in the arm of a chair, or an arm of a couch, or an arm-rest attached to, or placed near, a bed or other piece of furniture for seating or resting, positioned in such a way as to allow a subject to rest a finger, or fingers, or hand, or other body part for warming.

The surface of a warming plate may have one or more thermal controlled sites wherein the temperature of the target site on the patient (e.g., a finger) may be brought to a desired temperature. The temperature may be adjustable. By way of example and not limitation, embodiments may heat a finger or other target site to improve blood flow and thus blood yield from a finger-stick. For example, the warming plate may have thermal control areas to increase blood flow to the target area and thus increase the speed with which sufficient blood or other bodily fluid can be drawn from the subject. The heating derived from the heating element of a warming plate is used to bring the target tissue to between about 40° C. to about 50° C. In embodiments, the heating derived from the heating element is configured to bring target tissue to within a temperature range of between about 40° C. to about 44° C. In embodiments, the heating derived from the heating element brings target tissue to within a temperature range of between about 41° C. to about 43° C. In embodiments, the heating derived from the heating element brings target tissue to a temperature of about 42° C. In embodiments, the heating derived from the heating element brings target tissue to within a temperature range of between about 44° C. to about 47° C. In one embodiment, the temperature is sufficient to increase blood flow to yield about 120 uL of sample. In one embodiment, the temperature is sufficient to increase blood flow to yield about 130 uL of sample. In one embodiment, the temperature is sufficient to increase blood flow to yield about 140 uL of sample. In one embodiment, the temperature is sufficient to increase blood flow to yield about 150 uL of sample. Optionally, the thermal controlled site is a shaped surface is contoured to match that of the target site on a patient.

In embodiments, the thermal controlled surface of a warming plate comprises a hard surface that is easily cleaned, and that may be sterilized. Such a surface may be or include, for example, plastic, glass, hard rubber, acrylic, polymer, and other materials. In embodiments, the thermal controlled site comprises a soft or padded surface that is easily cleaned, and that may be sterilized. Such a surface may be or include, for example, cloth, rubberized cloth, soft rubber, and other materials.

When in use, the surface of the warming plate is typically at a higher temperature than ambient air temperature, and is effective to warm a fingertip placed thereon to between about 30° C. to about 46° C., or to between about 32° C. to about 45° C., or to between about 34° C. to about 44° C., or to between about 36° C. to about 43° C., or to between about 38° C. to about 42° C., or to between about 39° C. to about 41° C. In embodiments, the surface of a warming plate is maintained during use at a temperature of between about 30° C. to about 46° C., or of between about 32° C. to about 45° C., or of between about 34° C. to about 44° C., or of between about 36° C. to about 43° C., or of between about 38° C. to about 42° C., or of between about 39° C. to about 41° C.

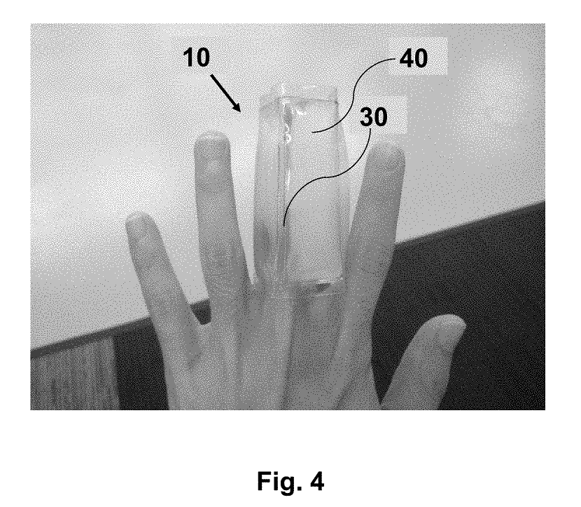

A finger warmer having features as illustrated in FIG. 4 may be, for example, activated to provide warmth and may be wrapped around a finger (or two or more fingers). The sample may be acquired after a minute, or two minutes, or three minutes, or more after wrapping. For example, following a few minutes of warming, the finger or fingers of the subject may be unwrapped sufficiently to expose a fingertip, and a lancet applied so as to pierce the skin and allow a few droplets of blood to escape the fingertip and be collected as a biological sample.

In one exemplary embodiment, a finger warmer having features as disclosed herein comprises a closed container enclosing a heating composition which can be triggered to provide heat. Thus, a finger warmer may comprise an enclosed compartment, having a flexible outer covering (e.g., a “skin”), such as a polymer, plastic, rubber, cloth, or other flexible material, enclosing materials which may, when desired, react in an exothermic reaction effective to release heat which may be used to warm an extremity of a subject. The flexible outer covering, or an inner portion of such a covering, is typically suitable for containing the enclosed materials without leakage, and is typically suitable for containing the enclosed materials in a state which prevents the occurrence of the exothermic reaction until it is purposefully triggered. Such an outer covering may be a water-proof covering, may be an air-tight covering, and, in embodiments, may be both water-proof and air-tight.

An outer covering of a finger warmer as disclosed herein is typically soft, or flexible, or both, and is typically configured to allow bending, folding, or other manipulation effective, e.g., to wrap a finger warmer around a finger, or around two or more fingers, of a subject. In embodiments, an outer covering of a finger warmer may comprise a polymer, such as Nylon®, Dacron®, Perlon®, Terylene®, Kevlar®, and other polymers or polymer blends. In embodiments, an outer covering of a finger warmer may comprise polyvinylchloride, polyethylene, polyurethane, or other polymer. In embodiments, an outer covering of a finger warmer may comprise a metallocene polymer, or a metallocene blend, such as, e.g., a metallocene nylon blend. In embodiments, a finger warmer may comprise a metallocene blend/nylon/metallocene blend composite. The perimeter of the outer covering, and/or select regions of interior of the warmer, may be fused together, e.g., by heat, or glue, or ultrasonic welding, or other means. In embodiments, a finger warmer may comprise an internal baffle, or a plurality of internal baffles, or may comprise two or more enclosed compartments. In embodiments, a finger warmer may be foldable, e.g., may be folded around an extremity of a subject, or folded around itself (folded up, as for storage). In embodiments, a finger warmer may be configured to be rolled, e.g., may be rolled around an extremity of a subject, or rolled around itself (e.g., rolled up, as for storage). In embodiments, a finger warmer may be flexible. In embodiments, a finger warmer may comprise hinges. Optionally, the finger warmer may have a substantially rectangular shape; the vertical lines within the rectangular perimeter indicate positions configured for folding the finger warmer. In embodiments, a finger warmer in an extended configuration may have a square, or round, or oval, or other shape.

Optionally, a finger warmer 10 may have a perimeter 20 that may be roughly rectangular in shape, with a length 12 of between about 3 to about 12 inches, or between about 4 to about 10 inches, or between about 4.5 and about 8 inches, or between about 5 and about 7 inches. A perimeter 20 may enclose an inner perimeter 22. In embodiments, a finger warmer 10 may have a length 12 of about 6 inches. A finger warmer 10 may have a width 14 of between about 1 and about 6 inches, or between about 2 and about 5 inches, or between about 2.5 and about 4 inches. In embodiments, a finger warmer 10 may have a width 14 of about 3 inches. A finger warmer 10 may include an outer seam, or weld, which may have a seam width 24 of between about 0.05 inches to about 0.5 inches, or between about 0.1 inches to about 0.3 inches, or of about 0.2 inches. A finger warmer 10 may thus provide an inner compartment 40, within the outer seam 24, having an internal width 16 of between about 0.5 and about 5 inches, or between about 1 and about 4 inches, or between about 1.5 and about 3 inches, or of about 2.5 inches.

A finger warmer 10 may include an internal baffle 30 or multiple internal baffles 30, which may facilitate folding a finger warmer by providing a thinner, or lower-resistance portion than other portions of the finger warmer 10. Internal baffles 30 may extend between one wall of the perimeter 20 and an opposite wall, or optionally, an internal baffle 30 may extend partially from one wall, stopping short of the opposite wall of perimeter 20. A first internal baffle 30 may be spaced apart from a second internal baffle 30, for example, by a spacing 36 of between about 0.3 to about 2 inches, or of between about 0.5 to about 1.5 inches, or of about 1 inch. An internal baffle 30 may have a baffle width 32 of between about 0.01 to about 0.5 inches, or between about 0.05 to about 0.2 inches, or may have a baffle width 32 of about 0.1 inches. An internal baffle 30 may have a baffle length 34 of between about 0.5 to about 6 inches, or may have a baffle length 34 of between about 1 to about 4 inches, or between about 1.5 to about 3 inches. In embodiments, an internal baffle 30 may have a baffle length 34 of about 2 inches.

A finger warmer 10 provides an enclosed space (e.g., internal compartment). In embodiments, an internal compartment 40 may comprise a volume of between about 5 mL to about 250 mL; or between about 10 mL and about 200 mL; or between about 15 mL and about 100 mL; or between about 20 mL and about 80 mL; or between about 30 mL and about 60 mL; or between about 35 mL and about 45 mL.

A finger warmer 10 may have a single internal compartment 40 (since, in this illustration, baffle length 34 is less than internal width 16, so that internal baffles 30 do not completely isolate individual portions of internal compartment 40). In embodiments, a finger warmer 10 may provide a plurality of enclosed spaces (e.g., enclosed spaces separated by barriers similar to internal baffles 30 but which extend completely from one perimeter wall to another, closing off one portion of an internal compartment 40 from another portion of an internal compartment 40).

An enclosed space (e.g., internal compartment) of a finger warmer 10 may hold a heating composition (e.g., a composition capable of providing heat, by, for example, an exothermic reaction). For example, the enclosed space of a finger warmer may hold a supersaturated salt solution (e.g., supersaturated sodium acetate in water) containing a trigger element, such as a metal disk which, when flexed, triggers crystallization of the salt, thereby releasing heat. In embodiments, the enclosed space of a finger warmer may hold a water-based solution including iron (typically as a powder), typically with a salt, and often also with activated carbon and other materials; the iron, when exposed to air, releases heat as the iron is oxidized, effective to warm a subject or a subject's extremity.

A finger warmer 10 may contain an oxidizable composition (e.g., a composition containing iron particles); or other composition which may release heat under proper conditions. An oxidizable composition may be contained or stored so as to prevent contact between the composition and air (or more particularly, between the composition and oxygen in the air); providing access to air initiates the desired heat generation. Thus, a finger warmer 10 containing an oxidizable composition within an internal compartment 40 may be maintained ready for use by preventing contact between the oxidizable composition and air, and heating may be initiated by allowing contact between the oxidizable composition and air (e.g., by removing a covering over a port which allows contact between the oxidizable composition and air, or otherwise breaching an air-tight enclosure containing the oxidizable composition).

A finger warmer 10 may contain a super-saturated solution, such as a super-saturated salt solution, which may release heat under proper conditions. A super-saturated salt solution (such as super-saturated sodium acetate) may include therein a trigger 50. A trigger 50 may be, for example, a metal disk which upon deformation is able to trigger the release of heat from the super-saturated solution. In embodiments in which a finger warmer 10 has a single internal compartment 40, activation of heating composition within the internal compartment 40 eventually activates all of the heating composition. In embodiments, such as embodiments in which an internal baffle 30, or multiple internal baffles 30, extend from one wall of perimeter 20 to an opposite wall of perimeter 20 to isolate one portion of internal compartment 40 from another portion of internal compartment 40, a finger warmer 10 may have separate portions of a heating composition enclosed in separate internal compartments 40. In such embodiments, in which a finger warmer 10 has multiple internal compartments 40, activation of heating composition within one internal compartment 40 may not activate the heating composition in another internal compartment 40, or may not activate the heating composition in all of the internal compartments 40.

In one exemplary embodiment of a finger warmer, a trigger 50 is shown in place within the internal compartment 40. A trigger 50 is used to initiate the release of heat from a heating composition within a finger warmer 10 (such initiation may be termed “activation” of the heating composition). For example, flexing or deformation of a trigger 50 within a super-saturated salt solution may be effective to initiate salt crystallization in the super-saturated solution; when the salt is an appropriate salt, such crystallization generates heat. For example, deformation of a trigger 50 within a super-saturated sodium acetate solution is able to trigger crystal formation in that solution. Without being bound by theory, it is believed that sodium acetate crystals formed in cracks, on peaks or discontinuities, or on the surface of the trigger 50 may be dislodged by deformation of the trigger 50, and so made available as nucleation centers for further sodium acetate crystal formation. In embodiments, a trigger 50 may be a packet or container holding sodium acetate (or other salt) crystals, and be able to initiate release of heat from a surrounding super-saturated sodium acetate (or other salt) solution by release of the crystals or other contact between the crystals and the super-saturated solution (e.g., by tearing or otherwise opening the packet or container).

In embodiments, a trigger 50 may comprise a metal disk, e.g., a thin metal disk having a thickness of between about 0.05 to about 0.4 inches, or between about 0.1 to about 0.3 inches, or of about 0.2 inches. In embodiments, a trigger 50 may comprise a thin metal disk having a diameter of between about 0.1 to about 2 inches, or of between about 0.3 to about 1.5 inches, or of between about 0.5 to about 1 inches, or of about 0.75 inches. In embodiments, a trigger 50 is typically made of a material which does not degrade or decay when in contact with sodium acetate or other salt. In embodiments a trigger 50 may be a stainless steel disk; in alternative embodiments, a trigger 50 may be made of another metal, or other material able to trigger an exothermic reaction when flexed or broken. In embodiments, a trigger 50 may have a square, or rectangular, or oval, or other shape. In embodiments, a trigger 50 may have a continuous surface, or may have holes, or pores, or other features breaking up the continuity of the trigger surface. In embodiments, a trigger 50 may have a flat surface, or may have an irregular surface, or a rough surface, or may include grooves, depressions, ridges, elevations, or other features rising from, or dropping into, the surface of a trigger 50.

Prior to activation of a heating composition in the internal compartment 40 of a finger warmer 10, the finger warmer 10 and its contents are typically at room temperature. Following activation of a heating composition, the finger warmer 10 and its contents may reach temperatures of between about 32° C. to about 48° C., or between about 35° C. to about 45° C., or between about 37° C. to about 43° C., or between about 39° C. to about 41° C. Heat generation to these levels may last for about 5 to 15 minutes, or about 8 to 10 minutes, following initiation of the heating process. If desired, the temperature range may be increased by alteration of the heating composition (e.g., by increasing the concentration of sodium acetate in a super-saturated sodium acetate solution, or by increasing the amount of concentration of iron in heating composition that relies on oxidation of iron to produce heat, etc.). Alternatively, lowering the concentration of salt or iron, or other active component of the heating composition may reduce the temperature range if less heat generation is desired.

Optionally, a finger warmer 10 may have fastener(s). Fasteners 60 and 70 may be used to secure a finger warmer 10 when the finger warmer 10 is rolled or folded around a finger. Fasteners may be, for example, Velcro® fasteners which, when in contact with each other, are able to hold a finger warmer 10 in a rolled or folded configuration around a finger of a subject. Fasteners may comprise different, complementary, fastener elements; for example where fasteners comprise Velcro® fasteners. In embodiments, a finger warmer 10 may have a strap, or belt, which may be used to hold a rolled or folded finger warmer 10 in a rolled or folded configuration, e.g., around a finger. Other details may be found in PCT Patent Application Ser. No. PCT/US14/54625 filed Sep. 8, 2014 and fully incorporated herein by reference for all purposes.

FIG. 4 shows a finger warmer 10 wrapped around the finger of a subject. The finger warmer 10 has been folded along baffles 30, and, as it is flexible, is also curved or flexed in other locations as well. Such folds, and such curvature, whether alone, or whether together, aid in wrapping a finger warmer 10 around a finger, or portion thereof, of a subject, and aid in warming the finger, or portion thereof, of the subject in order to facilitate acquisition of a biological sample from the subject.

### Hybrid Oversight

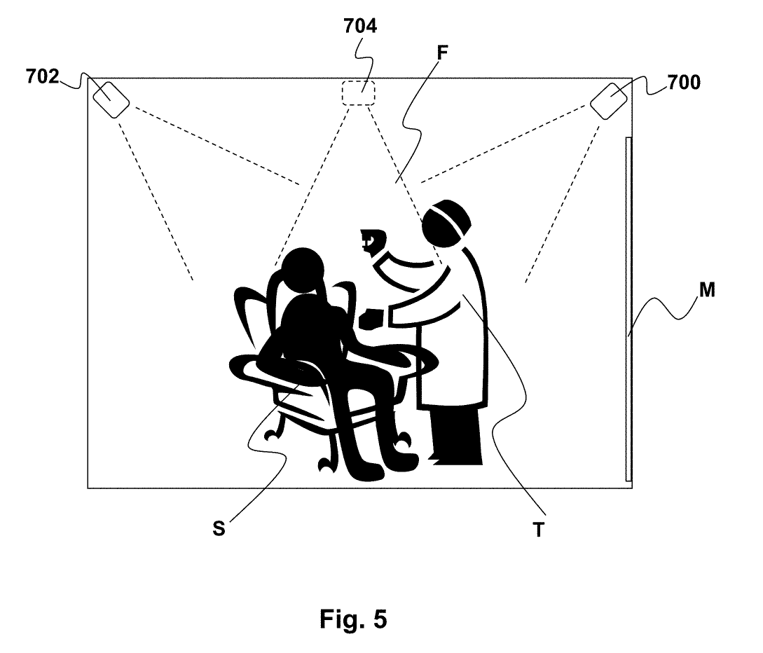

Referring now to FIG. 5, in one exemplary embodiment, a system can be used to provide oversight based at least in part on software using at least one imaging device such as but not limited to image and motion tracking systems such as but not limited to a Microsoft Kinect or similar motion tracking system 700 at a patient service center or other sample collection locations. Optionally, some embodiments may provide oversight primarily by using software for at least one image and motion tracking system such as but not limited to a Microsoft Kinect or similar motion tracking system, with optionally supplemental oversight using human review which may occur based on one or more alerts triggered by software-based oversight.

In one embodiment, the motion tracking system 700 functions by covering the room with a constant, predetermined pattern of infrared dots. A monochrome CMOS sensor may optionally be placed at an offset relative to the IR transmitter, and the difference between the observed and expected IR dot positions is used to calculate the depth at each pixel of the RGB camera.

In embodiments, the depth information is then used to calculate a skeletal model of any humans in view by running it through a Random Forest classifier, which classifies each pixel as being a body part (such as forearm or head), or background. Optionally, the features used is the set of distances in depth between groups of two pixels at a predetermined offsets from the pixel being classified. RGB camera data is not used for this, due to its high variability. Pixels corresponding to each body part are then clustered and fit to a skeletal model on a frame-by-frame basis, with no tracking occurring between frames. The RF classifier may be trained by taking a motion-capture dataset of human movements and augmenting it with artificial distortions to increase the training set.

Optionally, other embodiments may use multiple 3D imaging and/or motion tracking devices 700, 702, and 704. In one embodiment, the device 704 is overhead of the area of interest. Optionally, the device 704 is mounted on a side wall, wherein the device 704 is not directly overhead. Optionally, other embodiments may use multi-angle 3D imaging and/or motion tracking devices. Optionally, some embodiments may use an infrared projector, camera, and/or a microchip or processor to track the movement of objects and individuals in three dimensions. Optionally, some embodiments may use an RGB camera, depth sensor and multi-array microphone. Some embodiments of the system may be used to provide full-body 3D motion capture, facial recognition and voice recognition. Some embodiments may use at least one depth sensor comprising an infrared laser projector combined with a monochrome CMOS sensor, which captures video data in 3D under any ambient light conditions. In one embodiment, the distance of the detector to an area of interest may be in the range of about 11.5 ft. In one embodiment, the distance of the detector to an area of interest may be in the range of about 8 to 12 ft.

In one embodiment, the sensing range of the depth sensor is adjustable, and software associated with a motion tracking system such as but not limited to a Microsoft Kinect is capable of automatically calibrating the sensor based on gameplay and the player's physical environment, accommodating for the presence of furniture or other obstacles

In at least some embodiments, the movements for blood collection from a fingerstick is observable, and in many instances, should be predictable and repeatable. FIG. 5 shows a technician T obtaining a sample from a subject S. The process is one with certain variables, characteristics, actions, or identifiers which are verifiable for a certain set of these previously mentioned items and observable in the field of view F so the area on T being targeted by S. In one embodiment, the technician is trained to do the same things over and over. In many instances, ideally all of the technicians T are performing the collection in the same way to minimize risk that results in the lab test reflect variation due to the sample collection process.

Using one or more 3D imaging devices, one gets 3D views from at least two directions. In one embodiment, only one 3D view may not be sufficient. In such a configuration, the system can see 360 degrees by merging images from at least two of the 3D imaging devices. Optionally, some embodiments may use three or more imaging devices. Of course, using two Kinects or similar devices 700, one can get a 360 view. Optionally, some may use a mirror M on at least a portion of one wall with only one Kinect or similar device 700 that will allow for imaging of more than one side of a target area but using on a single device 700.

One embodiment may optionally put two Kinects or similar devices 700 viewing down at a target area. In one embodiment, software image analysis can then determine if the steps are being performed for a sufficient amount of time, in the right sequence, using the desired technique, or other variables.

Regarding the target area of interest, due to the size of the procedure room, the distance is such that quality imaging is possible, even with wall mounted imaging devices or devices mounted out of the field of patient-technician interaction. Although having imaging and/or motion detection devices closer to the patient is not excluded, it can be seen that such devices may also be mounted at a distance where their presence is more subtle and less obtrusive without significant compromise to successful completion of their task.

If a discrepancy is detected, feedback by a computer such as but not limited to screen alert, visual alert, voice alert, or the like, can notify the technician regarding the anomaly (and/or provide corrective instructions or other options). In some embodiments, this real-time feedback can approximate human oversight over the process by using a hybrid approach of providing automated monitoring when conditions are within normal ranges and then providing human oversight during certain discrepancy or uncertain periods when condition(s) are not in normal ranges.

In one non-limiting example, the setup can be configured to view the entire room or it can zoom into a desired area. Optionally, some embodiments may do at least both view the entire room and have an enlarged view of a desired area.

In one non-limiting example, the system can 1) recognize motions to see if the motions are correct (such as putting on a finger warmer). One movement that is well recognized is removing the finger warmer. Even if the technician attempts to hide the target area, instructions may be provided to the technician regarding the error condition, sound an alarm, and/or any corrective activity. This can be useful to prevent technicians from deviating from protocol or trying to trick the system.

In one non-limiting example, the system can also provide voice instructions as the procedure goes along (such as but not limited to please place fingerwarmer, 30 seconds has elapsed so please remove finger warmer, please discard fingerwarmer, please use alcohol to wipe target area, etc. . . . ). If the wrong finger is being wiped, then the computer can notify the technician. Optionally, the lancing device is also color coded and the system can detect if the desired color-coded device is being used. The training is consistent and the desired motion for these sample collection activities is limited so the system should be able to see it.

Many of these items are motion-capture related. In one embodiment, the system can provide prompts regarding what the subject is doing and what they should do next. Optionally, based on the technician's qualifications, the voice, feedback, or other oversight items can be reduced or toned down for more skilled practitioners. For example, the motions will still be observed but the amount of cueing and reminding may be limited. Accordingly, based on technician qualification, in some embodiments, the oversight can be designed to be more minimal. If an error is detected, an image is also saved for teaching/learning purposes.

Optionally, the collection procedure can also be recorded (specifically the erroneous portions) for review and so that the technician can view the error activity. If all went well, then the recording is not kept. If things go wrong, then the recording is kept. The recording may be a video recording. Optionally, the recording is a 3D or other multi-dimensional recording. Optionally, the recording is a series of still photographs. Optionally, the recording may include thermal, IR, or other imaging types. Optionally, errors captured on video can be archived for product development and for teaching.

In embodiments, the technician may wear certain clothing, gloves, or other distinctive or uniform gear to facilitate visualization by the motion tracking system 700. Optionally, even all or portions of the sample collection device may be marked in a manner to show orientation, direction, or location of key part(s) to facilitate visualization by motion tracking system 700. Some embodiments may use dots or shapes of fluorescent color on the technician or the collection device or even on the subject to facilitate tracking.

Optionally, some embodiments may include opt-in or opt-out options during appointment scheduling, other paperwork, or electronic signup form for subjects to see if they are willing to have their procedures preserved for educational, training, or other purposes. Optionally, some embodiments may blur or edit out the subject's face or other identifying area even after the subject is being recorded.

In one embodiment, the system may be configured to monitor a feature such as the angle of the angle (relative to horizontal or vertical) for a sample collection device during the sample collection event. In one non-limiting example, the angle check can be one feature. With imaging devices, angles and other variables can be confirmed as correct or not and noted in a database. Optionally, the system can also track progression of movement from acceptable zones to unacceptable zones.

In one embodiment, software and 3D motion detector may also be incorporated into the system. One can also attach motion capture badges, motion capture markers, or other indicators in the field of view or detection area of the system such as but not limited to being on the technician, a sample capture device, or the like so that their movements are observed more easily. One can also put motion detection on technician arm or other part of their person or clothing. Some embodiments may be configured for acoustics to determine distance. In one embodiment, the device 700 can do combinations of: video, audio, acoustics to see how far away you are, and motion capture.

Optionally, some other versions may include image analysis. Such image analysis, may for example, determine qualities regarding the sample being collected, the technician, or other information about the sample or the collection process. Image analysis may be used regarding the sample collection process, the sample collection device, and/or other area interest. Some embodiments may use a combination of motion detection with some image analysis.

Additionally, at least some embodiments of the system can also be used for training so that a trainee can get feedback and learn while doing. Real-time feedback to the technician can be provided. For example, the computer may say that the angle is wrong for your positioning. Optionally, one can also use this to train personnel at training centers before they are deployed into the field, not just oversight at an actual doctor's office or clinic. Technician could also wear ear piece to hear feedback. Optionally, feedback may be displayed on a technician's heads-up display, monitor, personal communication device, monocle, Google Glass device, visual display contact lens, wearable device, or other user-line-of-sight display device.

Optionally, the device(s) 700 only images a designated visible area (a colored desk, specific target areas, etc. . . . ). Optionally, the device 700 such as but not limited to a Kinect can have an angle detector in addition to image capture and/or motion capture capability.

In one embodiment, once a patient is gone from the procedure room, a score or other feedback may be given to technician through the system. Some may give it during procedure. Optionally, some other embodiments may be configured to wait until some other period or event such as the end of day or some other period to give grade or feedback to technician.

### Sample Collection

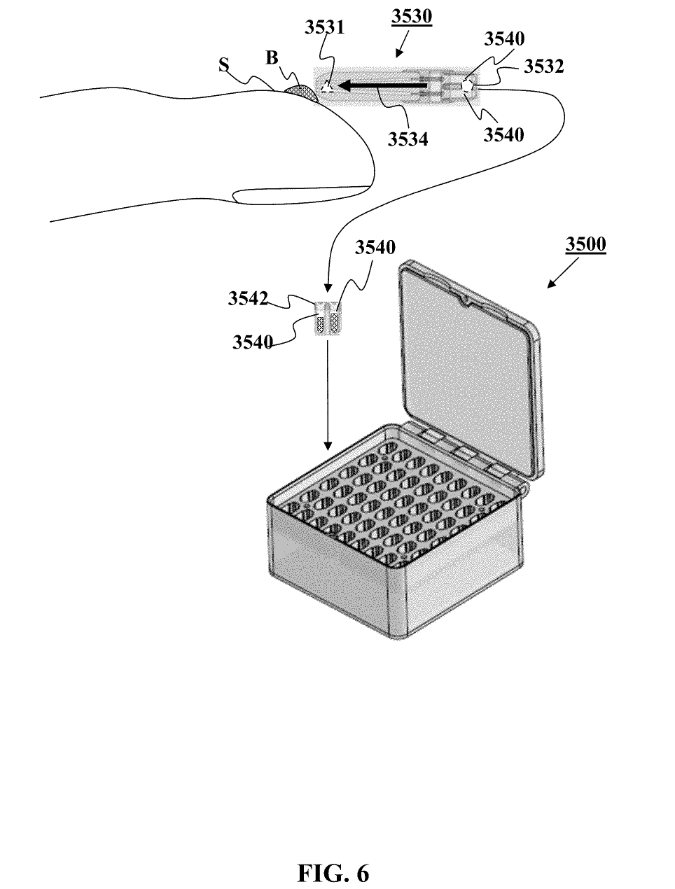

Referring now to FIG. 6, one embodiment of bodily fluid sample collection and transport will now be described. FIG. 6 shows a bodily fluid sample B on a skin surface S of the subject. In the non-limiting example of FIG. 6, the bodily fluid sample B can be collected by one of a variety of devices. By way of non-limiting example, collection device 3530 may be but is not limited to those described in U.S. Patent Application Ser. No. 61/697,797 filed Sep. 6, 2012, which is fully incorporated herein by reference for all purposes. In the present embodiment, the bodily fluid sample B is collected by one or more capillary channels and then directed into sample vessels 3540. By way of non-limiting example, at least one of the sample vessels 3540 may have an interior that is initially under a partial vacuum that is used to draw bodily fluid sample into the sample vessel 3540. Some embodiments may simultaneously draw sample from the sample collection device into the sample vessels 3540 from the same or different collection channels in the sample collection device. Optionally, some embodiments may simultaneous draw sample into the sample vessels

In the present embodiment after the bodily fluid sample is inside the sample vessels 3540, the sample vessels 3540 in their holder 3542 (or optionally, removed from their holder 3542) are loaded into the transport container 3500. In this embodiment, there may be one or more slots sized for the sample vessel holder 3542 or slots for the sample vessels in the transport container 3500. By way of non-limiting example, they may hold the sample vessels in an arrayed configuration and oriented to be vertical or some other pre-determined orientation. It should be understood that some embodiments of the sample vessels 3540 are configured so that they hold different amount of sample in each of the vessels. By way of non-limiting example, this can be controlled based on the amount of vacuum force in each of the sample vessels, the amount of sample collected in the sample collection channel(s) of the collection device, and/or other factors. Optionally, different pre-treatments such as but not limited to different anti-coagulants or the like can also be present in the sample vessels.

In embodiments, the sample collection device 3530 may include one or more markers, typically visual, that facilitate motion tracking by the system 700. By way of non-limiting example, some embodiments may have one or more motion tracking dots or shapes 3531 and 3532 that allow for showing orientation and/or position of the sample collection device. Optionally, these tracking shapes may be on at least one side, both sides of the collection device, top or bottom surfaces, front end surface, back end surface, all of the foregoing, or any single or multiple combination of the foregoing. Optionally, relative distance between the shapes can be used to indicate if the sample collection vessel has been activated, particularly when one shape is located on a body of the device and the second shape is on the movable sample collection vessel. Some embodiments may also use an orientation arrow or shape 3534 to show orientation of the sample collection device and indicate which end of the device is directed towards the subject. If the shape is a line or elongate shaped, it can also be used to see if the device is being held level or horizontal or if the device is being held at an angle relative to horizontal for the sample collection.

Optionally, some embodiments such as for capillary blood collection, may also monitor the start of collection when sample enters the collection device until the device is disengaged from the subject and sample is no longer being collected. For example, some embodiments may have pre-set times wherein sample collection should be completed. If the collection time exceeds this window, the system may tag the sample with the amount of time used for collection or mark the sample as rejected if the system cannot adjust calibration or other factors to accommodate the longer collection time. Optionally, some assays cannot use samples collected outside a desired window and thus if those assays are ordered, the database may compare the ordered assay with the collection time and then reject the sample for particular assays but still allow it for other(s) that may have been ordered but are not auto-rejected. In one non-limiting example, samples with collection times of more than 120 seconds is rejected. In one embodiment, collection time is measured from when the access site or wound is created on the subject until when sample is no longer being introduced/collected into the device. In one embodiment, collection time is measured from when the sample can be visualized on the subject (blood droplet appears on skin) until when sample is no longer being introduced/collected into the device. In one non-limiting example, samples with visually-observed collection times of more than 110 seconds is rejected. In one non-limiting example, samples with collection times of more than 100 seconds is rejected. In one non-limiting example, samples with collection times of more than 90 seconds is rejected. In one non-limiting example, samples with collection times of more than 80 seconds is rejected. Excess collection times for capillary blood samples (the actual time, the amount exceeding the allowed time, or some flag/indicator noting excess time) can be stored (automatically or by the technician's request) in the laboratory information system (LIS) or optionally EMR to provide addition information for sample integrity verification. Assay results may be adjusted based on calibrations for the longer collection time or the result rejected and not released if the time is too long. One example of an LIS system may be found in PCT Patent Application Ser. No. PCT/US14/48314 filed Jul. 25, 2014 entitled SYSTEMS AND METHODS FOR A DISTRIBUTED CLINICAL LABORATORY, hereby fully incorporated herein by reference for all purposes.

Optionally, there may also be technician profiles associated with the particular technician so that the motion tracking is tuned for that person. When the user is logged into the system, the appropriate user profile can be used with the motion tracking system 700 to better visualize motion tracking based on previous interactions with that technician. There may also be a calibration or tuning process where the motion tracking system can learn the motion associated with certain steps for that particular technician in the collection process. This may occur by way of the system recording an entire collection process or by the system 700 recording certain steps within the collection process where fine tuning of motion tracking is desired.

As seen in FIG. 6, the sample vessels 3540 are collecting sample at a first location such as but not limited to a sample collection site. By way of non-limiting example, the bodily fluid samples are then transported in the transport container 3500 to a second location such as but not limited to a receiving site such as but not limited to an analysis site. The method of transport may be by courier, postal delivery, or other shipping technique. In many embodiments, the transport may be implemented by having a yet another container that holds the transport container therein. In one embodiment, the sample collection site may be a point-of-care. Optionally, the sample collection site is a point-of-service. Optionally, the sample collection site is remote from the sample analysis site.

Although the present embodiment of FIG. 6 shows the collection of bodily fluid sample from a surface of the subject, other alternative embodiments may use collection techniques for collecting sample from other areas of the subject, such as by venipuncture, to fill the sample vessel(s) 3540. Such other collection techniques are not excluded for use as alternative to or in conjunction with surface collection. Surface collection may be on exterior surfaces of the subject. Optionally, some embodiments may collect from accessible surfaces on the interior of the subject. Presence of bodily fluid sample B on these surfaces may be naturally occurring or may occur through wound creation or other techniques to make the bodily fluid surface accessible. Although the sample collection device is shown in FIG. 6 at a horizontal orientation, it should be understood that other orientations can be used based on the desired collection procedure. Additional details regarding the device and sample collection process can be found in PCT application WO2014088606 filed Sep. 5, 2013 and PCT application PCT/US14/30792 filed Mar. 17, 2014, each application fully incorporated herein by reference for all purposes.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

Additionally, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a size range of about 1 nm to about 200 nm should be interpreted to include not only the explicitly recited limits of about 1 nm and about 200 nm, but also to include individual sizes such as 2 nm, 3 nm, 4 nm, and sub-ranges such as 10 nm to 50 nm, 20 nm to 100 nm, etc.

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications mentioned herein are incorporated herein by reference to disclose and describe the structures and/or methods in connection with which the publications are cited. The following applications are fully incorporated herein by reference for all purposes: U.S. Provisional Patent Application Ser. No. 62/085,229, filed Nov. 26, 2014 entitled Methods and Systems for Hybrid Oversight of Sample Collection.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. Any feature, whether preferred or not, may be combined with any other feature, whether preferred or not. The appended claims are not to be interpreted as including means-plus-function limitations, unless such a limitation is explicitly recited in a given claim using the phrase “means for.” It should be understood that as used in the description herein and throughout the claims that follow, the meaning of “a,” “an,” and “the” includes plural reference unless the context clearly dictates otherwise. For example, a reference to “an assay” may refer to a single assay or multiple assays. Also, as used in the description herein and throughout the claims that follow, the meaning of “in” includes “in” and “on” unless the context clearly dictates otherwise. Finally, as used in the description herein and throughout the claims that follow, the meaning of “or” includes both the conjunctive and disjunctive unless the context expressly dictates otherwise. Thus, the term “or” includes “and/or” unless the context expressly dictates otherwise.

This document contains material subject to copyright protection. The copyright owner (Applicant herein) has no objection to facsimile reproduction of the patent documents and disclosures, as they appear in the US Patent and Trademark Office patent file or records, but otherwise reserves all copyright rights whatsoever. The following notice shall apply: Copyright 2013-15 Theranos, Inc.

1. A method for sample collection from a subject, said subject having a body part, the method comprising:
obtaining, in a sample collection room, a sample from said body part of said subject;
monitoring activity in the sample collection room using at least two motion capture devices positioned to view a field of interest in the sample collection room; and
alerting a technician performing the sample collection if a non-conforming action by the technician is detected by at least one of the motion capture devices.

2. The method of claim 1 wherein alerting comprises providing instructions for at least one corrective action to the technician to remedy the non-conforming action.

3. The method of claim 1 wherein alerting further comprises connecting a human by way of a telecommunication link to the technician to guide the technician in at least one corrective action related to the non-conforming action.

4. The method of claim 2 wherein the corrective action comprises changing angulation of a sample collection device relative to horizontal.

5. The method of claim 2 wherein the corrective action comprises using a sample collection device with a different anti-coagulant therein.

6. The method of claim 2 wherein the corrective action comprises applying a warming device to a target tissue for a longer period of time.

7. The method of claim 2 further comprising attaching at least one motion capture reflective element to the technician.

8. The method of claim 2 further comprising attaching at least one motion capture reflective element to a garment to be worn by the technician.

9. The method of claim 2 further comprising providing an accessory to be worn by the technician, wherein said accessory comprises a motion capture reflective material.

10. The method of claim 2 further comprising providing at least one motion capture glove to be worn by the technician.

11. The method of claim 2 further comprising providing at least one motion capture finger marker to be worn by the technician.

12. The method of claim 2 further comprising providing at least one motion capture wrist marker to be worn by the technician.

13. The method of claim 2 further comprising providing at least one motion capture forearm marker to be worn by the technician.

14. The method of claim 2 wherein a sample collection device used for collection comprises at least one motion capture marker.

15. The method of claim 2 further comprising comparing motion captured by at least one of the motion capture devices with a reference motion stored for that technician when performing the sample collection.

16. The method of claim 2 further comprising comparing motion captured by at least one of the motion capture devices with a reference motion for performing the sample collection.

17. A sample collection motion tracking system.

18-28. (canceled)
